(19) United States
(12) Patent Application Publication
(10) Pub. No.: US 2003/0143628 A1
(43) Pub. Date: Jul. 31, 2003

(54) Potential profile generating method and protein tertiary structure prediction method and apparatus

(76) Inventor: Kentaro Onizuka, Kanagawa (JP)

Correspondence Address: MILLEN,WHITE,ZELANO,& BRANIGAN,P.C., 2200 Clarendon Boulevard, Suite l400, Arlington, VA, 22201, US

(21) Appl. No.: 10/239,861

(22) PCT Filed: Nov. 27, 2001
(86) PCT No.: PCT/JP01/10308

(51) Int. Cl. G01N 33/53; G06G 7/48; G06G 7/58

(52) U.S. Cl. 435/7.1; 703/11

(57) ABSTRACT

A method and apparatus for predicting a protein tertiary structure or designing a protein sequence using potential profiles calculated by using multidimensional singleton potentials dependent only on a residue type of one of residues of a residue pair and on a multidimensional relative structural relationship (including direction and orientation) between residues of each residue pair. Existing dynamic programming is used in predicting a protein tertiary structure.

### TECHNICAL FIELD

The present invention relates to a potential profile generating method, protein tertiary structure prediction method and apparatus, and protein sequence designing method, apparatus, program and storage medium.

### BACKGROUND ART

A protein that is the most important organism molecule for living beings is composed of one-dimensionally bonded smaller molecules called amino acids. A sequence of amino acids bonded one-dimensionally is called a protein sequence (or protein primary structure).

An “amino residue” is indicative of NH-CHRn.CO— that is a structural unit of an amino acid. Amino acids are combined by peptide bond; —CONH—.

In the specification, an amino residue is also referred simply to a “residue”. “Residue type” used in the specification means a type of an amino residue.

Actually, a protein molecule has a complicated tertiary structure, and exhibits a specific function by having such a tertiary structure. Accordingly, the function of a protein is determined by its tertiary structure.

Thus, clarifying the function of a protein requires clarifying its tertiary structure. It is possible to obtain a protein sequence of a protein in a short time by experiment.

Meanwhile, a tertiary structure is obtained by X-Ray Crystallography and/or Nuclear Magnetic Resonance (NMR) currently. However, it takes a few months to obtain a tertiary structure of a protein, and there are many proteins with known protein sequences but with unknown tertiary structures currently, requiring a technique for predicting a tertiary structure of a protein from a protein sequence of the protein.

In recent years there have been a few thousands types of proteins with known tertiary structures. As a result of structural class of proteins with known tertiary structures based on geometrical similarity, it has been understood that protein tertiary structures can be classified into less than 1000 types with insignificant differences neglected, and that even when protein sequences are greatly different, their tertiary structures may belong to the same structural class.

In such situations, with respect to a protein sequence of a given protein, predicting its tertiary structure is equal to finding out a structural class of protein known tertiary structure to which the tertiary structure belongs.

In other words, a protein sequence “s” of a protein targeted for tertiary structure prediction is compared with a plurality of tertiary structures already known, each compatibility is evaluated, and a tertiary structure with the highest compatibility is selected as a structure having the most similarity to a structure of the protein sequence.

Then, it is an issue how to evaluate the compatibility between a protein tertiary structure and a protein sequence.

One of conventional protein tertiary structure prediction methods is based on statistical physics and uses knowledge-based potentials of mean force (Sippl M. J. (1990) “Calculation of Conformational Ensembles from Potentials of Mean Force: An Approach to the Knowledge-based Prediction of Local Structure in Globular Proteins.” Journal of Molecular Biology, 213, pages 859-883). The knowledge-based potentials of mean force are obtained from a data set of known tertiary structures of proteins. By using the potentials, it is possible to calculate, as a sum of the potentials, the compatibility where a protein sequence is adapted to a tertiary structure. The availability of this method has been already established by a lot of calculations and experiments.

In one of the conventional protein tertiary structure prediction methods using mean-force potentials, calculation of mean-force potentials are based on both residue types of residues mutually acting on each other and a relative structural relationship between the residue types. The mean-force potentials calculated based on both residue types are called pairwise potentials.

In calculating pairwise potentials as in the conventional protein tertiary structure predicting protein tertiary structures, when calculating a mean-force potential of one (a) of residues at some position, it is necessary to specify a residue type of the other one (b) of residues. However, in a protein with an unknown tertiary structure, it is not possible to specify adjacent residues in the tertiary structure or specify effects of the residues, and therefore, it is not possible to specify a residue type of the other one (b) of residues. Thus, it is necessary to first calculate the alignment, and an algorithm is required to calculate evaluations of compatibilities using mean-force potentials to determine the alignment with the most excellent evaluation of compatibility.

Since there is an enormous number of alignment that a tertiary structure of a given protein likely has, it is not possible to evaluate all the compatibilities and calculate the most excellent evaluation of compatibility in a limited time. Accordingly, an algorithm is required that obtains the optimal alignment fast. However, such an algorithm that obtains the optimal alignment fast has not been known currently although various studies have been performed. There are some algorithms that have some limitations or obtain approximate solutions, but even those algorithms are not of fast algorithm.

Among such studies, Frozen approximation using pairwise potentials is used to fast obtain the alignment. In Frozen approximation, used as the other residue is a residue type of the other one (b) of residues corresponding to one (a) of residues in a tertiary structure used in the alignment. In the case of using Frozen approximation, by fixing a residue type of the other one of a pair of residues, it is made possible to generate a potential profile for each template protein targeted for identification. Then, it is possible to perform the alignment of a potential profile with a protein sequence of a protein (hereinafter, referred to as a prediction target protein) with an unknown tertiary structure, and to evaluate the compatibility. A dynamic programming is used in the compatibility evaluation.

However, in this method, when a protein sequence of the template protein is greatly different from a protein sequence of a prediction target protein, potentials absolutely different from pairwise potentials that are correct are used. Therefore, the structure identification accuracy deteriorates, and it is considered that this method is not capable of fast obtaining the optimal alignment with the identification accuracy kept.

### DISCLOSURE OF INVENTION

It is an object of the present invention to provide a protein tertiary structure prediction method and apparatus, and pertinent program and storage medium capable of predicting protein tertiary structures with high accuracy and at high speed. It is another object of the present invention to provide a protein sequence designing method and apparatus capable of determining a protein sequence with a desired structure at high speed and with high accuracy, and a computer readable storage medium with therein stored a program for the designing method.

As a result of considerable efforts, the inventor of the present invention found out that in a protein tertiary structure prediction method, by using multidimensional mean-force potentials, it is possible to predict a protein tertiary structure using singleton potentials with structure prediction accuracy equal to or more than that in using pairwise potentials.

Conventionally, in a practical protein tertiary structure prediction method, when calculating mean-force potentials, only a distance (hereinafter referred to as a residue-distance) between residues (a) and (b) is used as a relative structural relationship between the residues. This is called one-dimensional potential.

The mean-force potentials are assumed to inherently reflect physicochemical properties of residues in a protein, in particular, the hydrogen bond of residues spaced (a distance) in a sequence and interaction between side chains of residues. Therefore, it is naturally considered that the potentials are not dependent only on a distance, but are dependent on overall relative structural relationship.

Therefore, by considering a direction (hereinafter referred to as a relative direction) and an orientation (hereinafter referred to as a relative orientation) of one of residues from the other one of residues, as a relative structural relationship between the residues, in addition to a distance between the residues, it is expected to further approximate the mean-force potentials to the physicochemical potentials, and to improve prediction performance in the protein tertiary structure prediction method using mean-force potentials.

Thus, the above mean-force potential is called a multi-dimensional potential which is calculated using the relative structural relationship between residues including the relative direction and relative orientation in addition to the distance between residues, which the inventor of the present invention has proposed in “A compressed representation of multi-dimensional distribution by linear base-transformation, and its application to the residue-pair relative distribution of proteins, Kentaro Onizuka, Tamotsu Noguchi, Makoto Ando, and Yutaka Akiyama, Information Processing Society of Japan, Vol. 40, No. SIG2 (TOM1), pages 105 to 116 (1999), [D-98-135]). By using thus obtained multi-dimensional potentials, it has been expected to further approximate the mean-force potentials to the physicochemical potentials, and to improve prediction performance in the protein tertiary structure prediction method using mean-force potentials.

Actual mean-force potentials have been calculated based on a degree distribution with respect to both residue types, separation distance and residue-distance, which is statistically examined. In this case, a residue-distance is divided, for example, into segments with 1 Å, and the number of samples present in each segment is counted. In the case of using such a method (binning method) of calculating the degree distribution in segments, it is necessary to divide the direction and orientation also into segments when achieving the mean-force potentials with the direction and orientation considered in addition to the residue-distance. For example, in the case of dividing each into ten bins a distance, zenithal angle and longitude among a direction, and three Euler angles as an orientation, there are total one million small bins, and it is necessary to count the number of samples in each of one million small bins. In other words, the number of parameters obtained as statistics reaches one million orders. However, the number of proteins whose tertiary structures are already known is actually a few thousands, and the number of pairs obtained from samples of such proteins as residue pairs having a specific residue type and separation distance among residue pairs in the same protein is only a few hundreds to a few thousands. Therefore, although the number of samples is almost a few hundreds, the number of bins is one million and the number of parameters to be obtained is one million. Thus it is impossible to calculate stable statistics.

As described above, the multi-dimensional mean-force potential is expected to improve the prediction performance theoretically, but has not been applied as a practical protein tertiary structure prediction method, because in actual statistics, results are not satisfactory and advantages overcoming such an inconvenience that the statistical calculation is complex has not been found.

Besides aforementioned multi-dimensional potentials, the inventor of the present invention has examined using singleton potentials instead of pairwise potentials. The singleton potential is obtained based on a single residue type among two (or more) residues composing potentials. Accordingly, since the singleton potential is not dependent on a residue type (b) of the other residue of the pair, the energy can be calculated when a residue type (a) targeted for the calculation is only known.

Further, by averaging residue types of the other residue of the pair, it is made possible to generate a potential profile for each protein of a learning data set, perform the alignment of the potential profile with a protein sequence targeted for the tertiary structure prediction, and to evaluate the compatibility. Furthermore, there are advantages such that dynamic programming algorithm is used in compatibility evaluation.

In the method using singleton potentials, potentials are averaged with respect to residue types of the other residue of the pair. Therefore, the structure identification accuracy deteriorates, and it is considered such a method is not able to obtain the optimal alignment fast with the identification accuracy kept.

The inventor has searched for an algorithm capable of obtaining alignment with high accuracy and at high speed in a protein tertiary structure prediction method, and found out introducing multidimensional potentials to compensate for defects of singleton potentials while taking advantage of singleton potentials.

In other words, as described above, it has been considered that adopting singleton potentials instead of pairwise potentials results in inadequate performance because potentials are averaged on the other residue type (b) even though there is an advantage such that only identification of one residue type (a) is sufficient for calculation. However, it has been found out that even in the case of using singleton potentials, by using multidimensional potentials instead of one-dimensional potentials based on a residue-distance, it is possible to generate a potential profile without using complicated algorithms, use a more general and fast dynamic programming algorithm in evaluating compatibilities, and to obtain performance equal to the case of using pairwise potentials.

The present invention has been carried out based on such knowledge.

That is, in the present invention, in obtaining energy values for each residue type in each of residue positions in a protein tertiary structure that is already known and generating a potential profile composed of information of the energy values for each residue type in each of residue positions, multidimensional singleton potentials are used as potentials used in obtaining the energy values, where each of the singleton potentials is dependent on a residue type of a single residue among two or more residues associated with the potential, and is further dependent on a relative direction and relative orientation between the residues.

In other words, in the case of considering a pair of residues interacting with each other and calculating the mean-force potential of one of the residues, the singleton potentials are adopted that are independent of residue type of the other one of the residues, thereby facilitating and hastening the calculation processing.

Meanwhile, by expanding the mean-force potential from one-dimensional potential dependent only on a distance to multidimensional potential dependent also on a relative direction and relative orientation between residues, the accuracy in description by the mean-force potential is enhanced, thereby overcoming accuracy deterioration caused by adopting singleton potentials.

In other words, by adopting the multidimensional singleton potentials, it is possible to secure accuracy higher than that in potential in Frozen approximation described previously using pairwise potentials (potentials calculated with residue types of a pair of residues specified).

An aspect of the protein tertiary structure prediction method of the present invention includes a step of obtaining a frequency distribution of multidimensional relative structural relationship of each of all residue pairs of each protein from tertiary structure data of a plurality of proteins with known tertiary structures, a step of by using the frequency distribution, with respect to each position of residues of the residue pairs of each template protein, calculating an energy value based on multidimensional singleton potentials dependent on the multidimensional relative structural relationship and only on a residue type of one of residues of a residue pair from the tertiary structure data of a plurality of proteins with known tertiary structures, and adding energy values for each residue type to obtain mean-force potentials, and a step of by using the calculated mean-force potentials, evaluating a compatibility between a protein sequence of a protein with an unknown tertiary structure to be predicted and a protein sequence of each template protein, and searching for a template protein having a tertiary structure similar to that of the prediction target protein.

In another aspect of the protein tertiary structure prediction method of the present invention, in a similar way to conventional methods, so-called structure identification is performed in which based on a protein sequence of a protein (prediction target protein) with an unknown structure, a protein having a tertiary structure similar to the prediction target protein is searched from proteins (template proteins) with known tertiary structures.

In the present invention, it is first required to prepare in advance groups (hereinafter referred to as learning data set) of tertiary structure data (which represents three-dimensional positional coordinates of all atoms included in a molecular of each protein) of proteins with known tertiary structures.

For example, the learning data set is available from tertiary structure database of thousands of proteins with known tertiary structures (for example, registered with Protein Data Bank (PDB) operated by Research Collaboratory for Structural Bioinformatics (http://www.rcsb.org/pdb/)).

Further, it is possible to select tertiary structures having adequate verities and no redundancies from the above protein tertiary structure database. Considered as a selecting method is a method for selecting data based on the homology of protein sequence so that a plurality of data with similarities higher than some level is not contained to avoid redundancy, or a method of selecting typical tertiary structure data with higher accuracy from each structural class category based on tertiary structure class.

In the present invention, in order to first calculate mean-force potentials, with respect to each protein composing the prepared learning data set, the relative structural relationship “s” of each residue pair is calculated from the tertiary structure data, and its frequency distribution fak(S) is obtained and stored.

Frequency distribution fak(s) is indicative of that with respect to a residue type “a” of one of residues, what kind in distance, direction and orientation of the other residue (spaced k in a sequence) is distributed around the residue type “a”.

Specifically, for each protein, the relative structural relationship “s” of one pair of residues “a” and “b” is calculated. The relative structural relationship “s” is represented in multi-dimension (two to six) including at least a residue-distance “r” among residue-distance “r”, relative directions θ and φ, and relative orientations θe, φe and φe, preferably represented in multi-dimension composed of a combination of residue-distance “r” and at least one of relative directions θ and φ, and relative orientations θe, φe and φe, and most preferably represented in three-dimensional composed of residue-distance “r” and relative directions θ and φ.

In the case of representing the relationship “s” in this three-dimension, relative structural relationships of residue pairs of a protein are separated almost completely, and it is not likely that different relative structural relationships have the same distance and direction.

Next, frequency distribution (frequency statistics) fak(s) is obtained from relative structural relationships “s” of all pairs of residues “a” and “b” for each protein in the learning data set.

In order to obtain frequency distribution fak(s), as multidimensional frequency statistical processing, for example, an information compressing operation using Fourier expansion is performed. In this case, the relative structural relationship “s” of each pair of residues “a” and “b” is integrated with linear base and transformed into expansion coefficients, and the coefficients are added, for example, for the entire protein, each residue type “a” or each separation distance “k”, and the added value is obtained as expansion coefficient aI. For all proteins composing the learning data set, expansion coefficients aI are calculated and stored.

By using the information compressing operation using Fourier expansion, it is possible to reduce the number of parameters that has increased to million orders due to the multidimensional processing to hundred orders. More specifically, the frequency distribution fak(s) of relative position relationship “s” is expanded with orthonormal linear base gI(s) in a space in which the relative structural relationship “s” is present. In other words, as shown in equation (1), the frequency distribution fak(s) is represented by expansion coefficients aI. Herein, “I” represents an expansion order. Since it is multidimensional expansion, “I” is a vector composed of expansion order in each dimension.

fak(S)=Σ_{I}a_{I}g_{I}(S) (1)

Since the frequency distribution fak(s) is originally obtained from samples, when each sample is assumed to be δ function (delta-function) with respect to the relative structural relationship “s” of the sample, the frequency distribution fak(s) is a total sum of δ function (s−si) with each sample assumed to be “si”. In other words, fak(s) is given by equation (2).

fak(S)=Σ₁δ(s−sᵢ) (2)

Since gI(s) is an orthonormal base, gI(s) meets orthonormal conditions expressed by following equations (3-a) and (3-b).

When I is not equal to J, ∫g_{I}(s)g_{I}(s)ds=1 (3-a)

When I is equal to J, ∫g_{I}(s)g_{I}(s)ds=0 (3-b)

Accordingly, the expansion coefficient aI is obtained from fak(s) and gI(s) from following equation (4), and is a total sum of values of expansion base function on samples.

a_{I}=∫g_{I}(s)fak(s)ds=∫g_{I}(s)Σᵢδ(s−sᵢ)ds=Σᵢg_{I}(s) (4)

When the frequency distribution fak(s) is represented by thus obtained aI, the representation accuracy is dependent on how much the expansion is performed. It is not possible to calculate a number of expansion coefficients exceeding the number of samples to obtain significant statistics. When the relative structural relationship “s” is of one-dimensional value, the number of expansion coefficients (or the number of cut-off orders) indicates resolution in representation of fak(s). However, in the case of multi-dimension, in the same way as the image compression method using DCT (Discrete Cosine Transform), by devising the cut off, it is possible not to decrease the resolution even with expansion coefficients decreased.

According to this method, even when the number of apparent expansion coefficients is small, it is possible to maintain the high accuracy in representation.

The generalized information compressing operation using Fourier expansion is applied to statistics of the mean-force potential between residues of a protein as described below.

As shown in FIG. 6, since CA, N and C atoms form a triangle with an almost same shape independent of residue type and condition and it is thereby possible to define local coordinates, relative positions of paired residues “a” and “b” interacting with each other are perfectly determined from a position (represented by three-dimensional coordinates) of the other residue “b” in a local coordinate specific to one residue “a” and orientation (three-dimensional rotation angles, in general, represented by Euler angles θe, φe and φe) of the other residue “b” with respect to one residue “a”.

For the position of the other residue “b” viewed from one residue “a”, three-dimensional polar coordinates γ,θ and φ are used instead of Cartesian coordinate because ad. For the relative orientation, general Euler angles θe, φe and φe) are used.

As linear bases in Fourier expansion, for radius component r, a normalized Spherical Bessel-Neumann function is used which is used as a radius component of a wave function in potentials having sphere symmetry used in, for example, the quantum theory. Let the function with an order of i be Ri (r). For direction components θ and φ and orientation components φe and φe, normalized Spherical harmonics Ylm (θ,φ) are used. For orientation component θe, trigonometric functions sin nθe and cos nθe are used. Accordingly, orthonormal gI (=gijklmn; γ,θ,φ,θe, φe, φe) associated with the relative structural relationship “s” with six parameters, i.e., distance γ, directions θ and φ, and orientations θe, φe and φe, is a product of orthogonal bases normalized by respective parameters, and is expressed as following equations (5-a) and (5-b).

gᵢⱼₖₗₘₙ(γ,θ,φ,θe, φe,φe)=CᵢⱼₖₗₘₙRᵢ(γ)Yⱼₖ(θ,φ)sin nθeYₗₘ(φ,φ) (5-a)

gᵢⱼₖₗₘₙ(γ,θ,φ,θe,φe,φe)=CᵢⱼₖₗₘₙRᵢ(γ)Yⱼₖ(θ,φ)cos nθeYₗₘ(φ,φ) (5-b)

Herein Cᵢⱼₖₗₘₙ is a normalizing constant.

In terms of performance and expansion-order cut off, it is not required always to calculate statistics on all the six parameters. When necessary, only three parameters γ,θ and φ may be used. Various methods are considered for expansion-order cut off. For example, it is set that the total sum of expansion orders i, j, k, l, m, n for parameters does not exceed a predetermined value.

The method as described above enables multidimensional mean-force potentials, and a protein tertiary structure prediction method using such potentials drastically improves the performance.

In the present invention, in the information compressing operation using Fourier expansion, as linear bases of a distance direction component, it is preferable to use Legendre Polynomial that is orthonormal in a designated area.

In the case of representing multidimensional potentials by polar coordinates, as described above, the spherical Bessel-Neumann function is used for Fourier expansion base Ri(r) in radius direction r. However, for two reasons that expansion regions are determined as described below and there is no periodicity, it is preferable to use normalized Legendre polynomial Pi(z).

In this case, since distance r is expanded in a range from minimum rmin to maximum rmax, variable transformation between r and z is defined as expressed in following equations (6-a) to (6-b) so that the range accords with (−1, 1) that is an orthogonal region in Legendre polynomial.

Rᵢ(r)=Pᵢ(Z) (6-a)

Z=2(r−rₘᵢₙ)/(rₘₐₓ−rₘᵢₙ)−1 (6-b)

In this way, with respect to the radius component, statistics are computed in a range of “r”, from rmin to rmax, and therefore, and when the same number of bases is used to expand, the special resolution in the statistics in radius direction is enhanced as compared with the method using spherical Bessel-Neumann function where statistics are computed in a range of r from 0 to a specific distance rmax. As a result, it is possible to provide fine mean-force potentials. Actually, it is known that residue-distance “r” is more than 3 Å except adjacent residues. For rmax, for example, values ranging from 10 Å to 20 Å are applied when necessary.

The case of calculating fak(s) using Fourier expansion is as described above. In the present invention, it may be possible to generate a histogram and calculate frequency distribution fak(s) using the histogram. In other words, in order to represent the multidimensional distribution, a multidimensional space on which the distribution is present is divided into fine spaces (bins), the number of samples present in each fine space is counted, and the distribution is represented by the number of samples in fine space. However, in the case of high dimension, since the number of fine bins is of million orders as described above, when the number of samples is of a few hundreds to a few thousands orders, there are a large amount of fine spaces in which samples are not present and the statistics in such a case is insignificant.

In a protein tertiary structure prediction method according to the present invention, potential profiles are generated for each protein from the frequency distribution fak(s) of learning data set calculated and stored as described above. The alignment of the potential profiles with a protein with an unknown tertiary structure is performed to evaluate the compatibility described later.

The potential profile is generated as described below. First, with respect to all pairs of residues “a” and “b” in tertiary structures of a plurality of template proteins with known tertiary structures to be identified, energy values (also called compatibility evaluation values) ΔEak(s) based on mean-force potentials for twenty residue types “a” are calculated from the frequency distribution fak(s) restored from expansion coefficients aI. Energy values (potential values) Pia in residue position i are calculated for each of the twenty residue types. The energy values Pia are added for each residue type to obtain the compatibility evaluation value ΔE(S, C). These are collectively called a potential profile.

The potential profiles are obtained and stored for all the template proteins.

In the present invention, in generating the potential profiles, the multidimensional potentials are used which are dependent on relative directions and relative orientations besides residue-distances, instead of using one-dimensional potentials which are dependent only on residue-distances.

In other words, since the energy value ΔEak(s) is obtained from expansion coefficients aI calculated from the relative structural relationship “s” represented in multi-dimension, the energy value is of a potential with the same dimension amount as aI calculation.

Further in the present invention, instead of using pairwise potentials dependent on residue types of both paired residues “a” and “b” (“b” is one or more), singleton potentials are used which are dependent only on one residue type “a” among paired residues “a” and “b”.

In other words, since the energy value ΔEak is obtained and add for a pair of one residue “a” and residue “b” (one or more) present in a range of a predetermined distance rmax in which the residues can be paired, the energy value is of the singleton potential which is dependent only on one residue “a” with potentials averaged for different paired residues “b”.

The alignment of the potential profile generated as described above with a protein sequence of the prediction target protein is performed, the compatibility between the potential profile and the protein sequence is evaluated, and a protein with a tertiary structure similar to that of the prediction target protein is selected from template proteins.

The compatibility evaluation will be described more specifically. In the compatibility evaluation, the compatibility evaluation value ΔE(S,C) when a protein sequence of a prediction target protein is applied to a tertiary structure of a template protein is obtained from the potential profile, and the optimal alignment is obtained that provides the most excellent compatibility evaluation value ΔE(S,C) between protein sequence S and tertiary structure C.

In such compatibility evaluation, as an algorithm to obtain the optimal alignment, a dynamic programming algorithm is used that is generally known and works fast.

In the compatibility evaluation using the dynamic programming, a residue type is read one by one in turn starting from one end of a protein sequence of a prediction target protein, a compatibility evaluation value (=energy value=score) of the read residue type in a corresponding residue position in the potential profile is read and added. The total of compatibility evaluation values of residues from one end to the other end is obtained (hereinafter, the total of compatibility evaluation values is referred to as “alignment score”).

Such processing is executed for potential profiles obtained from all the template proteins. Then, alignment results are presented of proteins corresponding to potential profiles with higher alignment scores. From among the results, users using the protein tertiary structure prediction method select tertiary structures suitable in biology and chemistry, and based on them, predicts a tertiary structure of the prediction target protein.

As described above, in the present invention, in the protein tertiary structure prediction method using mean-force potentials, the mean-force potentials are set to be singleton potentials dependent only on one residue type “a” of the residue pair, and the mean-force potentials are expanded from one-dimensional potentials based only on residue-distance to multidimensional potentials. Therefore, it is possible to overcome the defect of singleton potential, i.e., deterioration of accuracy in structure identification caused by averaging potentials of the other residue “b”, and to obtain the same accuracy as in using pairwise potentials.

In using singleton potentials, the compatibility evaluation value of each residue in each residue position in a tertiary structure of a template protein is determined independently of a protein sequence of a prediction target protein and alignment with the sequence.

Accordingly, at a stage where a plurality of proteins is selected as learning data set, it is possible to generate a potential profile in advance for each protein tertiary structure.

Therefore, it is not required to calculate energies when a protein sequence of a prediction target protein is given, and to perform tertiary structure prediction only by performing alignment of the given sequence with stored potential profiles. Further, at a stage of the alignment, the entire energy is also calculated and optimized. As a result, it is possible to greatly reduce a time taken for protein tertiary structure prediction.

Potential profiles can be generated also in the case of using Frozen approximation using pairwise potentials. However, as described above, in the case where protein sequences are greatly different between a prediction target protein and a template protein, the reliability in the alignment extremely deteriorates.

Further, after obtaining the alignment, it is necessary to perform so-called remount where the total sum of pairwise potentials is calculated based on the alignment to calculate the entire energy value. Furthermore, it is sometimes necessary to perform optimal evaluation while changing the alignment using a so-called repeating method in which the energy value ΔEabk is repeatedly calculated (the remount is repeated) so as to obtain its accurate value until the alignment is converged, using a result of the alignment obtained in Frozen approximation as a new template. It takes a considerable time also to perform this method.

According to the present invention, it is possible to use the evaluation value of alignment by the dynamic programming as an energy value for the compatibility evaluation of the alignment of a protein sequence with the potential profile, and it is not required to perform the remount.

As described above, according to the present invention, it is possible to implement optimal alignment of a protein sequence of a protein with an unknown tertiary structure with a tertiary structure of a protein to be identified using general and fast dynamic programming, and to remarkably enhance the accuracy in structure identification.

Using multidimensional potentials in a protein tertiary structure prediction method has been proposed already, but has not obtained satisfactory effects on actual statistics. One of subject matters of the present invention is to compensate for defects of singleton potential to take advantages thereof. Thus, according to the present invention, it is found out that benefits are obtained from a different point of view, and multidimensional potentials are first put in practical use.

Further, in the present invention, as described above, using singleton potentials enables the use of dynamic programming algorithm in compatibility evaluation. The issue of this case is what evaluation value (gap scoring) is given to insertion and lack (hereinafter also referred to as a gap). In the case of using mean-force potentials, there are some methods of calculating suitable scoring for the insertion and lack, but the scoring cannot be calculated strictly and is only given an approximate value. Generally, a method is empirically used of giving a poor evaluation value (gap penalty) to insertion and lack to subtract points.

In general, when there is insertion or lack in a protein sequence, a considerably poor evaluation value (first gap penalty) is given as a penalty and a linear poor evaluation value (extension gap penalty) is given to a length of insertion or lack. However, in this method, alignment is not stable due to a value of penalty of insertion or lack. Further, in the case where the same protein sequence is aligned with protein tertiary structures with different lengths, the alignment score is dependent on the number of residues of the tertiary structure, and it is not possible to calculate the normalized score.

Then, in order to overcome the above problems, the inventor of the present invention noticed that in the dynamic programming, adding a good evaluation value (hereinafter, also referred to as a continuation bonus) corresponding to a length of a portion without gap (insertion or lack) on a path enables the continuousness of nodes without gap to be evaluated, and it is thereby possible to obtain compatibility evaluation scale that is stable also when lengths are remarkably different between a tertiary structure and protein sequence.

In the dynamic programming, as described above, when a protein sequence is aligned with a potential profile, correspondence is found of portions with a good compatibility evaluation value (high compatibility) between the sequence and potential profile, and a gap is assigned to a portion of a sequence or potential profile without correspondence. In this case, since assigning a gap decreases the evaluation value corresponding to the gap, a poor compatibility evaluation value is given to such a portion. In other words, a penalty is imposed on a gap.

In the present invention, in addition to the penalty, when there is a continuous matching area where portions match continuously without a gap, a good compatibility evaluation value, i.e., bonus is given. More specifically, in the present invention, in a two-dimensional matrix (with a protein sequence on one side and potential profile on the other side) in the dynamic programming, when selecting an optimal path from a plurality of paths of other nodes merging with a node, scores of determined optimal paths of other nodes from an origin are compared, and a path of one of other nodes with the highest score is selected. At this point, in the case where a path proceeding in a slanting direction is continuous on the two-dimensional matrix, which indicates that a portion of a protein sequence matches with a portion of a potential profile on the determined path, a node on the continuous path is given a bonus.

In this way, since a total sum of compatibility evaluation values is increased on the alignment with less gaps, the evaluation is dependent on the number of gaps independently of all the lengths of gaps as compared with a conventional case where a penalty is given to a gap, and the alignment is obtained so that corresponding portions are as large as possible over the whole. Experimental results proved that this method has an effect of obtaining preferable alignment while avoiding insertion or lack.

In the compatibility evaluation by the dynamic programming of the present invention, such a method (hereinafter referred to as continuation bonus method) for adding a continuation bonus may be combined with a gap subtracting method for giving a penalty to a gap.

This evaluation method is capable of being used widely for general alignment of DNA and residue, in addition to the protein tertiary structure prediction method of the present invention using singleton potentials as described above.

Further in the compatibility evaluation, the alignment is performed between a protein sequence with an unknown tertiary structure and potential profile, and when an ith residue in the protein sequence matches with a jth residue of a template (potential profile), an energy value of the jth residue (mean-force potential) is added as a score. Such a score is local one for the jth residue, it is called a local compatibility evaluation value.

In the present invention, preferably, instead of the local compatibility evaluation value, an average of energy values (mean-force potentials) of the jth residue and a plurality of residues in the vicinity of the jth residue are added as a score. The score is called a vicinity compatibility evaluation value on the jth residue.

In the case of using the vicinity compatibility evaluation value, averaging energy values in the vicinity introduces the compatibility of adjacent residues, and thereby provides a stable local compatibility evaluation value even when the local compatibility evaluation value is low accidentally (which often occurs in protein), and as a result, the alignment is stabilized. Independently of how to impose a penalty on a gap, similar alignment is always obtained. This evaluation method enables optimal alignment to be obtained using the dynamic programming even when a sequence permits a gap.

An aspect of a protein tertiary structure predicting apparatus of the present invention has a frequency distribution calculating section that obtains a frequency distribution of multidimensional relative structural relationship of each of all residue pairs of each protein from tertiary structure data of a plurality of proteins with known tertiary structures, a mean-force potential calculating section which with respect to each residue position of residue pairs of each template protein, calculates an energy value based on multidimensional singleton potentials dependent on the multidimensional relative structural relationship and only on a residue type of one of residues of a residue pair, from tertiary structure data of a plurality of template proteins to be identified with known tertiary structures, using the frequency distribution obtained in the frequency distribution calculating section, and adds energy values for each residue type to obtain mean-force potentials, and a compatibility evaluating section that performs compatibility evaluation between a reside sequence of a prediction target protein with an unknown structure and each of the template proteins.

The processing for predicting protein tertiary structures of the present invention is primarily divided into a data preparing step of obtaining frequency distributions from a learning data set to generate potential profiles of template proteins, and a structure predicting step of searching for a template protein with a similar structure by performing compatibility evaluation between a protein sequence and potential profile. The steps of the processing are capable of being carried out independently of each other.

In other words, the technical scope of the present invention includes a potential profile generating method and apparatus for generating potential profiles from a learning data set to predict tertiary structures.

The potential profile generating method of the present invention for evaluating a compatibility between a protein sequence of a prediction target protein with an unknown structure and each of template proteins to search for a template protein with a tertiary structure similar to that of the prediction target protein includes a step of obtaining a frequency distribution of multidimensional relative structural relationship of each of all residue pairs of each protein from tertiary structure data of a plurality of proteins with known tertiary structures, and a step of, with respect to each residue position of residue pairs of each template protein, calculating an energy value based on multidimensional singleton potentials dependent on the multidimensional relative structural relationship and only on a residue type of one of residues of a residue pair, from tertiary structure data of a plurality of template proteins to be identified with known tertiary structures using the frequency distribution and adding energy values for each residue type to obtain mean-force potentials, and obtaining mean-force potentials for each template protein to generate potential profiles.

The potential profile generating apparatus of the present invention for evaluating a compatibility between a protein sequence of a prediction target protein with an unknown structure and each of template proteins to search for a template protein with a tertiary structure similar to that of the prediction target protein has a distribution calculating section that obtains a frequency distribution of multidimensional relative structural relationship of each of all residue pairs of each protein from tertiary structure data of a plurality of proteins with known tertiary structures, and a potential profile generating section which with respect to each residue position of residue pairs of each template protein, calculates an energy value based on multidimensional singleton potentials dependent on the multidimensional relative structural relationship and only on a residue type of one of residues of a residue pair, from tertiary structure data of a plurality of template proteins to be identified with known tertiary structures, using the frequency distribution, adds energy values for each residue type to obtain mean-force potentials, and obtains mean-force potentials for each template protein to generate potential profiles.

The technical scope of the present invention further includes a protein tertiary structure prediction method and apparatus for preparing already generated potential profiles, and using the profiles, predicting a tertiary structure of a protein from a protein sequence of the protein.

That is, a protein tertiary structure prediction method of the present invention includes the steps of evaluating a compatibility between a potential profile using multidimensional singleton potentials dependent on a multidimensional relative structural relationship between residues obtained from a frequency distribution of known protein tertiary structures and only on a residue type of one of residues of a residue pair, and a protein sequence of a prediction target protein with an unknown structure, and searching for a template protein having a tertiary structure similar to that of the prediction target protein based on the evaluation result.

The present invention further provides a protein tertiary structure predicting apparatus comprised of a compatibility evaluating section that evaluates a compatibility a between a potential profile using multidimensional singleton potentials dependent on a multidimensional relative structural relationship between residues obtained from a frequency distribution of known protein tertiary structures and only on a residue type of one of residues of a residue pair, and a protein sequence of a prediction target protein with an unknown structure, and a similar tertiary structure searching section that searches for a template protein having a tertiary structure similar to that of the prediction target protein based on the evaluation result.

The present invention further includes a program for implementing the above-mentioned protein tertiary structure prediction method and a computer readable storage medium storing the program.

That is, the present invention provides a program for making a computer execute a procedure of obtaining a frequency distribution of multidimensional relative structural relationship between residues of each of all residue pairs of each protein from tertiary structure data of a plurality of proteins with known tertiary structures, a procedure of, with respect to each residue position of residue pairs of each template protein, calculating an energy value based on multidimensional singleton potentials dependent on the multidimensional relative structural relationship and only on a residue type of one of residues of a residue pair, from tertiary structure data of a plurality of template proteins to be identified with known tertiary structures using the frequency distribution, and adding energy values for each residue type to obtain mean-force potentials, and a procedure of by using stored mean-force potentials, evaluating a compatibility between a protein sequence of a prediction target protein with an unknown structure and each of the template proteins to search for a template protein likely having a tertiary structure similar to that of the prediction target protein.

Further, the present invention provides a program which is used in evaluating a compatibility between a protein sequence of a prediction target protein with an unknown structure and each of template proteins to search for a template protein likely having a tertiary structure similar to that of the prediction target protein, and which is used in making a computer execute a step of obtaining a frequency distribution of multidimensional relative structural relationship between residues of each of all residue pairs of each protein from tertiary structure data of a plurality of proteins with known tertiary structures, and a step of, with respect to each residue position of residue pairs of each template protein, calculating an energy value based on multidimensional singleton potentials dependent on the multidimensional relative structural relationship and only on a residue type of one of residues of a residue pair, from tertiary structure data of a plurality of template proteins to be identified with known tertiary structures using the frequency distribution and adding energy values for each residue type to obtain mean-force potentials, and obtaining mean-force potentials for each template protein to generate potential profiles.

Furthermore, the present invention provides a program for making a computer execute the procedures of evaluating a compatibility between a potential profile using multidimensional singleton potentials dependent on a multidimensional relative structural relationship between residues obtained from a frequency distribution of known protein tertiary structures and only on a residue type of one of residues of a residue pair, and a protein sequence of a prediction target protein with an unknown structure, and of searching for a template protein having a tertiary structure similar to that of the prediction target protein based on the evaluation result.

Still furthermore, the present invention provides computer readable storage media storing the above programs.

Moreover, the present invention provides, with respect to proteins with desired structures (i.e., designed structures), protein sequence designing method, apparatus and program, and storage medium storing the program.

### BRIEF DESCRIPTION OF DRAWINGS

FIG. 1 is a block diagram illustrating a configuration of a protein tertiary structure predicting apparatus of a first embodiment of the present invention;

FIG. 2 is a flow diagram illustrating statistical processing procedures in a statistical processing section in the protein tertiary structure predicting apparatus in the first embodiment;

FIG. 3 is a flow diagram illustrating procedures of generating a potential profile in a potential profile generating section in the protein tertiary structure predicting apparatus in the first embodiment;

FIG. 4 is a flow diagram illustrating procedures of evaluating a compatibility in a compatibility evaluating section in the protein tertiary structure predicting apparatus in the first embodiment;

FIG. 5 is a diagram to explain a method of adding a bonus in alignment in the protein tertiary structure predicting apparatus in the first embodiment;

FIG. 6 is a diagram illustrating a relative structural relationship between paired residues “a” and

FIG. 7 is a block diagram illustrating a configuration of a protein sequence designing apparatus according to a second embodiment of the present invention;

FIG. 8 is a diagram to explain a protein sequence determining method;

FIG. 9 is a flow diagram illustrating procedures of the protein reside sequence determining method;

FIG. 10A is a diagram to explain a method of acquiring mean-force potential information using multidimensional singleton potentials;

FIG. 10B is a diagram to explain potential profile generation using multidimensional singleton potentials;

FIG. 11 is a diagram to explain processing for performing optimal alignment of a protein sequence having an unknown tertiary structure with a template protein having a known tertiary structure, using generated potential profiles;

FIG. 12 is a diagram to explain processing for selecting a template protein predicted to have a most similar tertiary structure from a plurality of template proteins;

FIG. 13 is a flow diagram to explain procedures of generating a potential profile;

FIG. 14 is a flow diagram to explain a protein tertiary structure prediction method;

FIG. 15 is a block diagram illustrating a protein sequence designing apparatus according to the second embodiment of the present invention; and

FIG. 16 is a flow diagram illustrating procedures of designing a protein sequence.

### BEST MODE FOR CARRYING OUT THE INVENTION

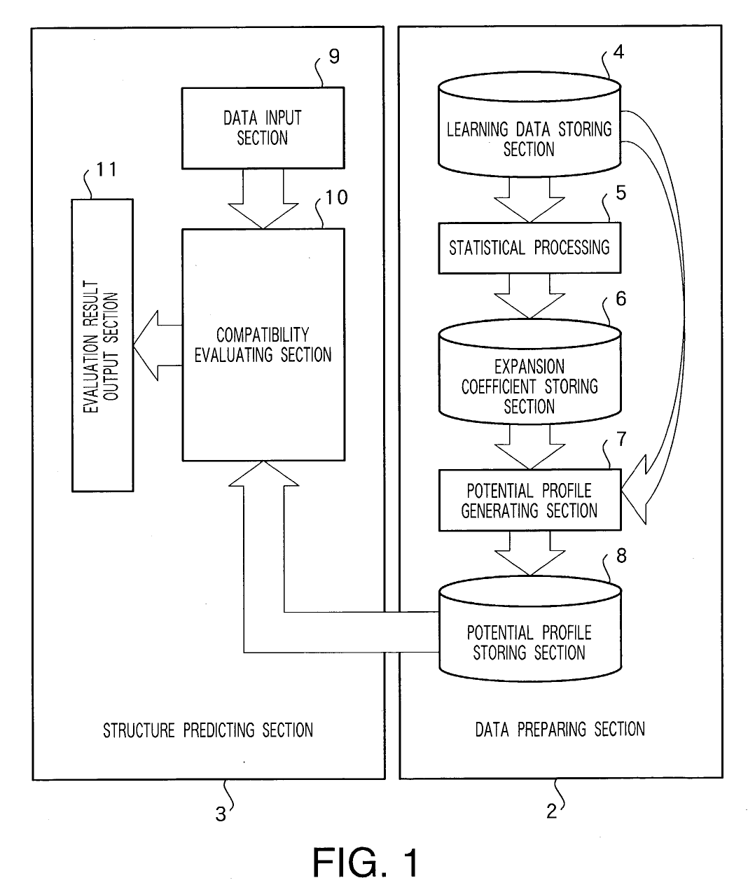

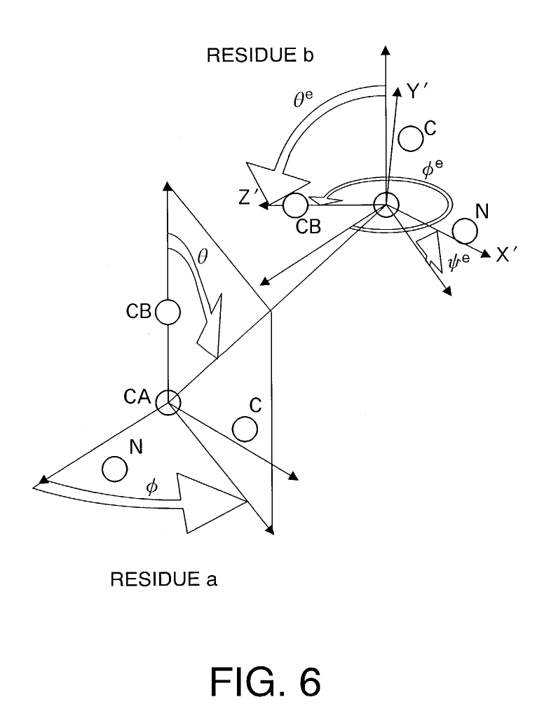

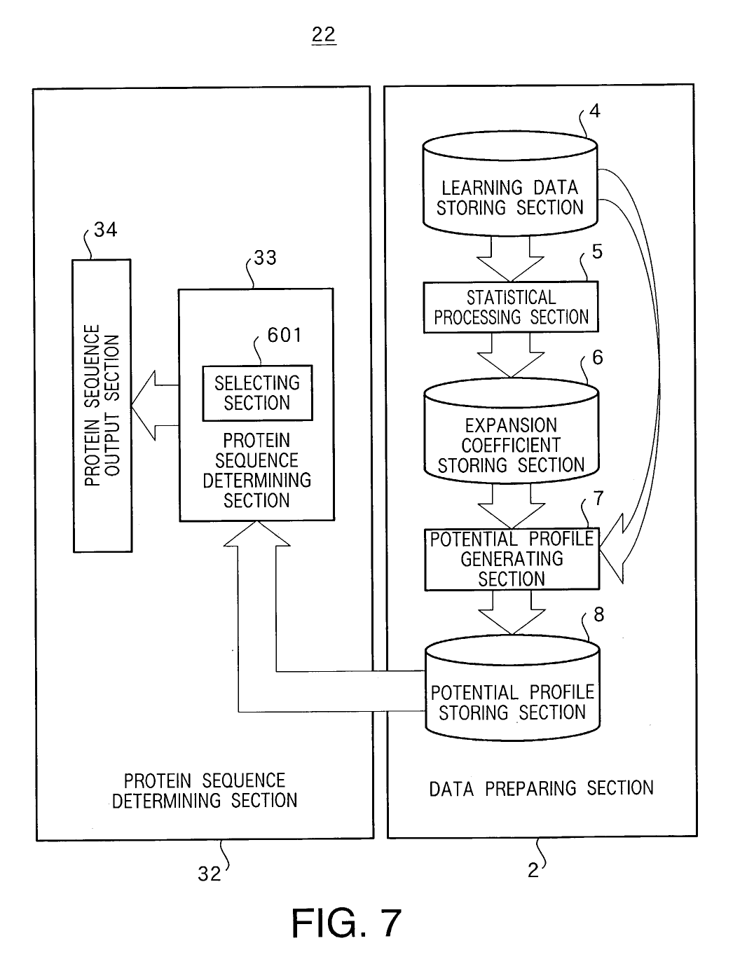

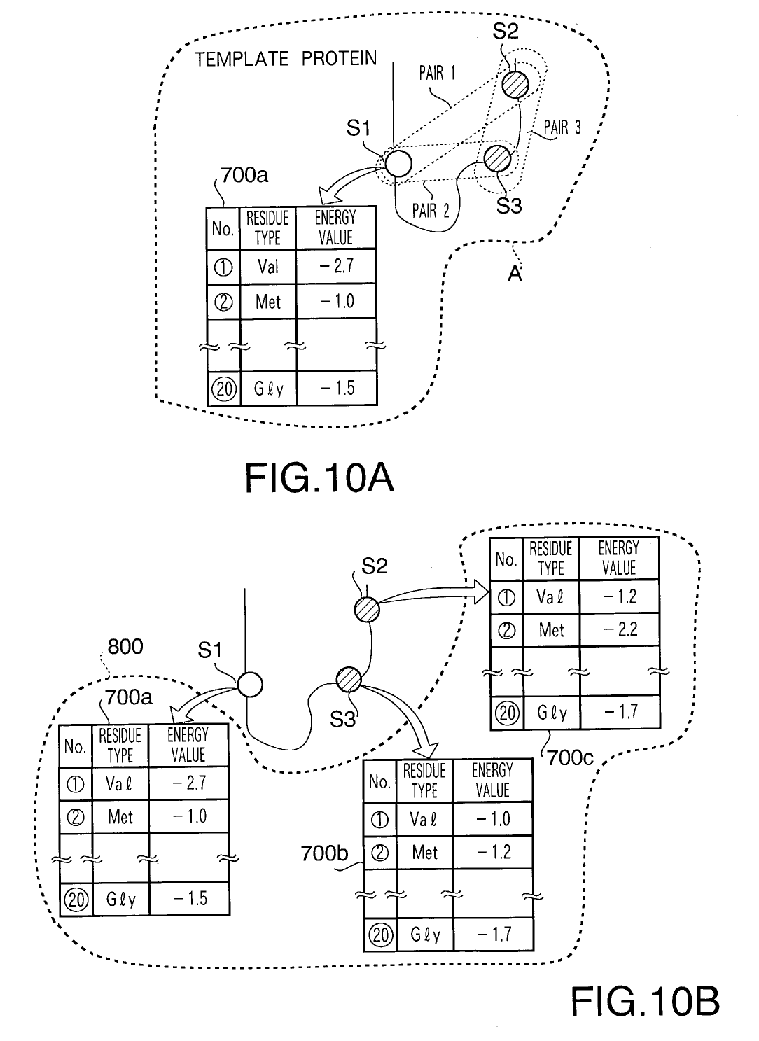

A summary of the present invention will be described first with reference to FIGS. 10 to 16. Then, the first embodiment (technique for predicting protein tertiary structures) will be described with reference to FIGS. 1 to 6, and the second embodiment (technique for designing protein sequences) will be described with reference to FIGS. 7 to 9.

First, potential profiles will be described with reference to FIGS. 10A and 10B. In the following description, an extremely simplified convenient model is used for easy understanding.

A protein is assumed which has a known tertiary structure as illustrated in FIG. 10A and has three amino residues, i.e., S1, S2 and S3, at respective residue positions (positions with a high probability of the residue being present).

In the structure of the protein, three pairs are assumed, i.e., a pair of S1 and S2 (referred to as pair 1), a pair of S1 and S3 (referred to as pair 2) and a pair of S2 and S3 (referred to as pair 3).

Residues have effects on each other. For example, an energy value of residue S1 is expressed by a sum of an energy value (mean-force potential) calculated based on pair 1 and an energy value (mean-force potential) calculated based on pair 2.

When calculating mean-force potentials for each pair using conventional pairwise potentials, it is necessary to specify each residue type of paired residues, and to perform operations on all likely combinations, resulting in an enormous amount of operations.

On the contrary, in singleton potentials used in the present invention, for example, when potentials on residue S1 are calculated, since types of the other residues (S2 and S3) are not involved in the operations, it is only required to consider a residue type of residue S1, and it is possible to obtain with simplicity energy values at each residue position without fixing a protein sequence, resulting in greatly simplified operation processing.

However, since accuracy (reliability) of the energy value using singleton potentials decreases, the present invention aims to increase the accuracy of the energy value by providing singleton potentials with multidimensional parameters corresponding to relative direction between residues (including relative orientation when necessary).

It is thereby possible to obtain potential profiles with high reliability in consideration of only type of residue at each residue position and relative direction and orientation between paired residues, using a simple method.

By using such potential profiles with high reliability, it is possible to predict protein tertiary structures with high reliability and to design protein sequences of designed proteins rapidly with accuracy In the case of calculating energy values on residue S1, energy values are calculated for each residue type. There are twenty types (for example, Valine (Val), Methionine (Met), Glycin (Gly), etc.) of amino acids composing proteins, and energy values are calculated for each of the types.

As described above, energy values are obtained by adding mean-force potentials obtained for each predicted pair.

The energy values include information indicative of potential likelihood at a residue position, and therefore, referred to as “mean-force potential”.

In this way, obtaining energy values (mean-force potentials) for each of twenty residue types on residue S1 acquires potential information 700a on residue S1 as illustrated in FIG. 10A.

Then as illustrated in FIG. 10B, the similar operations are performed for residues S2 and S3, and potential information 700b and 700c is obtained respectively for residues S3 and S2.

Mean-force potential information at all residue positions in a protein tertiary structure is calculated, whereby potential profile 800 as illustrated in FIG. 10B is generated.

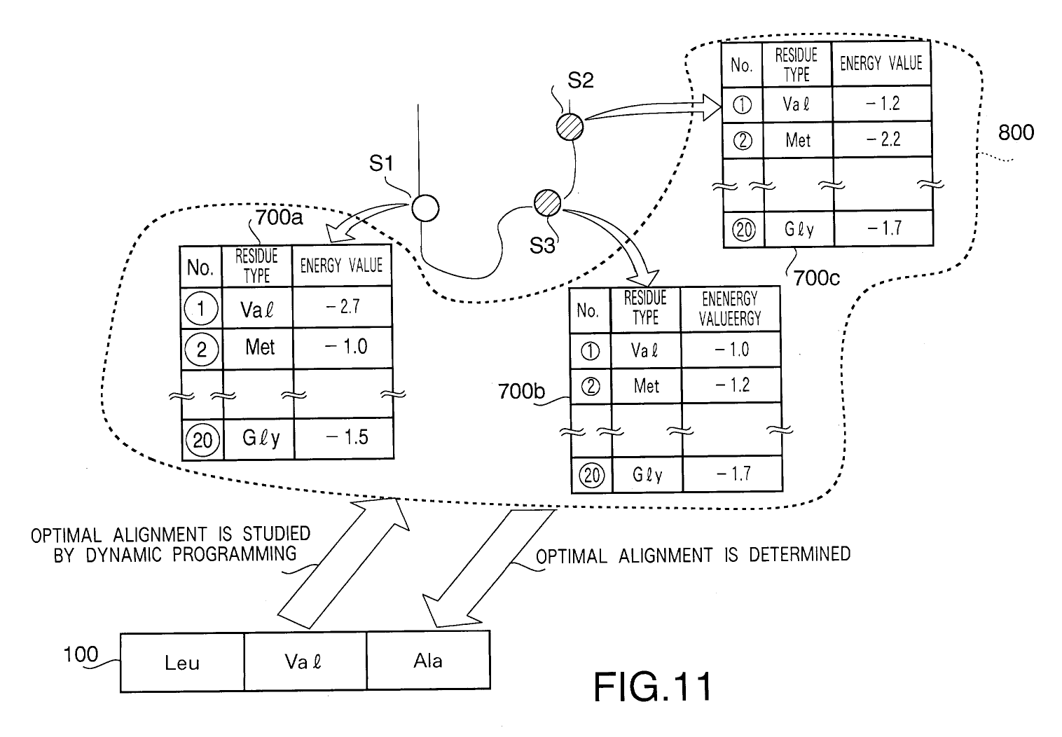

In the first embodiment of the present invention, as illustrated in FIG. 11, a potential profile is generated in advance on a template protein with a known tertiary structure, and using the profile, optimal alignment (arrangement of residues in a sequence) is performed on protein sequence 100 with a known sequence and unknown structure using the dynamic programming.

The optimal alignment is basically determined so as to minimize a total energy value of energy values at respective residue positions.

The total energy value for the optimal alignment is assumed to be an alignment score (evaluation value) representing the template protein.

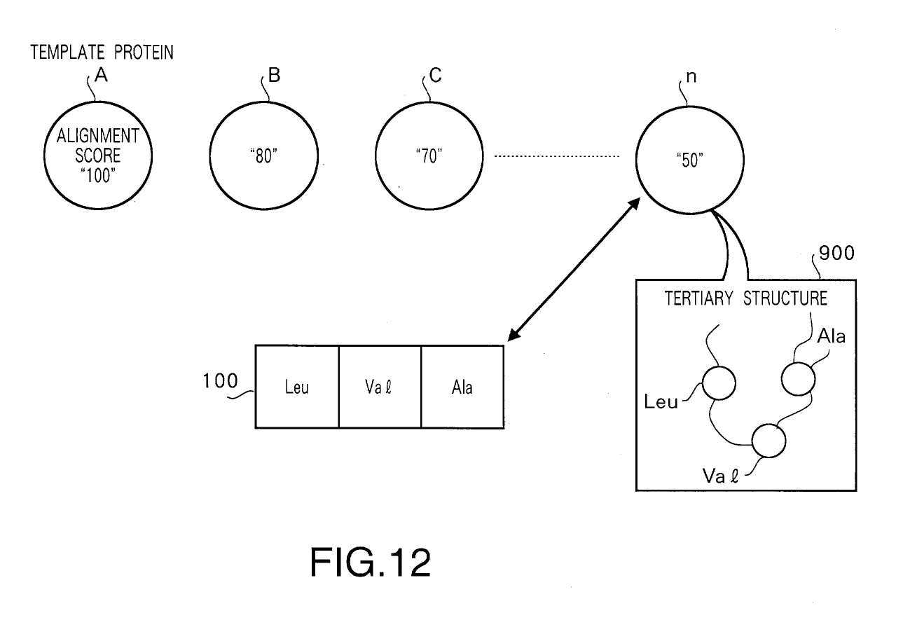

Then, as illustrated in FIG. 12, for example, alignment scores of optimal alignment results of a plurality of template proteins A to n are compared to each other to select a template protein with the smallest score (in FIG. 12, template protein n is selected), and a tertiary structure of the template protein is predicted to be the most likely structure of protein sequence 100.

In addition, in FIG. 12, Leu denotes Leucine that is one of amino acids composing proteins in organisms, Val denotes Valine, and Ala denotes Alanin.

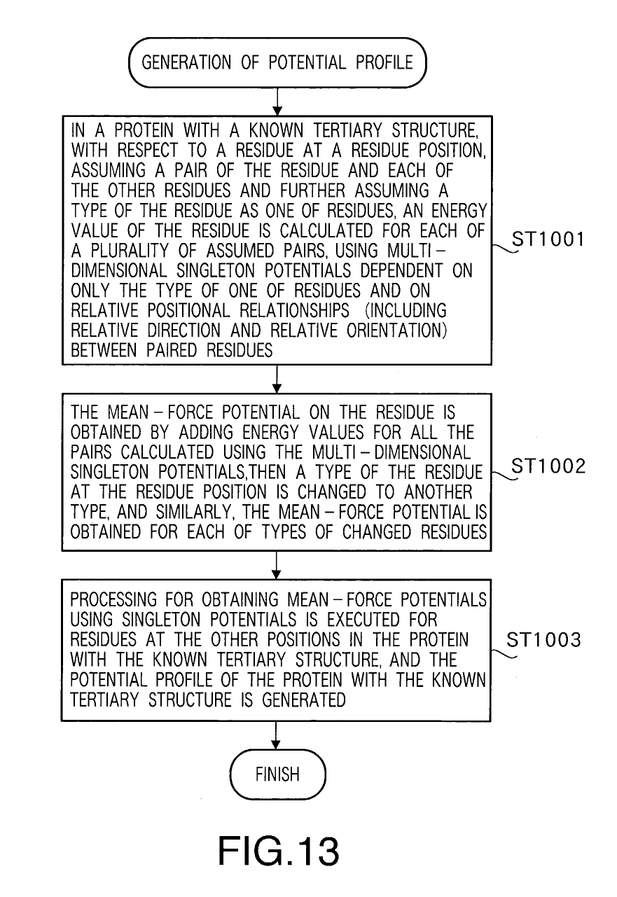

Procedures of processing for generating a potential profile as described above are as illustrated in FIG. 13.

In a protein with a known tertiary structure, with respect to a residue at a residue position, assuming a pair of the residue and each of the other residues and further assuming a type of the residue as one of residues, an energy value of the residue is calculated for each of a plurality of assumed pairs, using multidimensional singleton potentials dependent on only the type of one of residues and on relative structural relationships (including relative direction and relative orientation) between paired residues (ST1001).

The mean-force potential on the residue is obtained by adding energy values for all the pairs calculated using the multidimensional singleton potentials, then a type of the residue at the residue position is changed to another type, and in the same way as the foregoing, the mean-force potential is obtained for each of types of changed residues (ST1002).

The processing for obtaining mean-force potentials using singleton potentials is executed for residues at the other positions in the protein with the known tertiary structure, and the potential profile of the protein with the known tertiary structure is generated (ST1003).

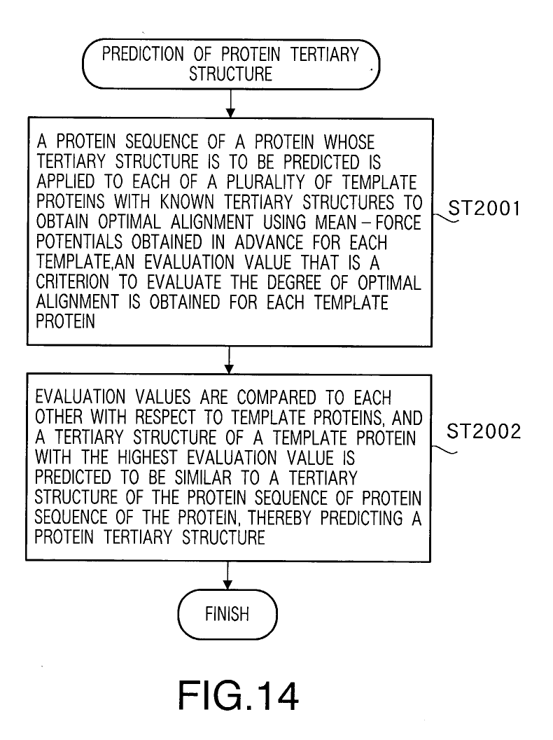

Further, procedures for predicting a protein tertiary structure are as illustrated in FIG. 14.

A protein sequence of a protein whose tertiary structure is to be predicted is applied to each of a plurality of template proteins with known tertiary structures to obtain optimal alignment using mean-force potentials obtained in advance for each template, an evaluation value that is a criterion to evaluate the degree of optimal alignment is obtained for each template protein (ST2001).

Evaluation values are compared to each other with respect to template proteins, and a tertiary structure of a template protein with the highest evaluation value is predicted to be similar to a tertiary structure of the protein sequence of the protein, thereby predicting a protein tertiary structure (ST2002).

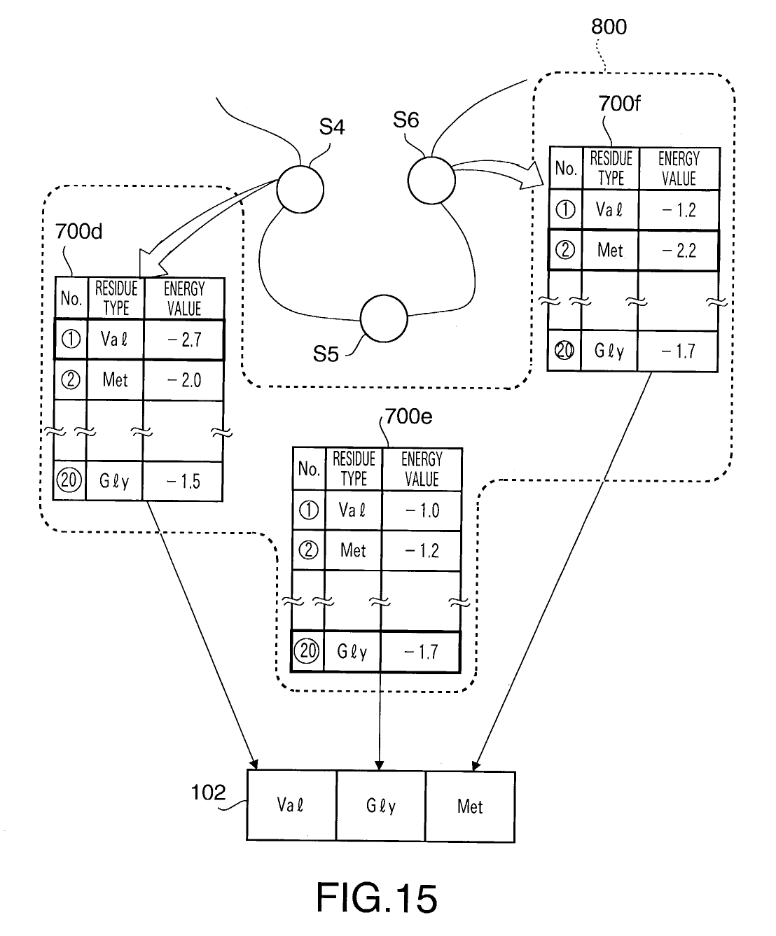

Further, in the second embodiment of the present invention, as illustrated in FIG. 15, a protein sequence of a protein with a designed structure is determined using potential profiles.

As illustrated in FIG. 15, when assuming a protein with a designed structure (in other words, having a desired structure) and with an unknown protein sequence and determining the protein sequence with residue types S4, S5 and S6 specified, mean-force potential information 700d, 700f and 700e at respective residue positions is acquired, a residue type with the lowest mean-force potential is selected at each residue position, and thus a desired protein sequence is uniquely determined. In this way, it is also possible to design protein sequences rapidly and accurately.

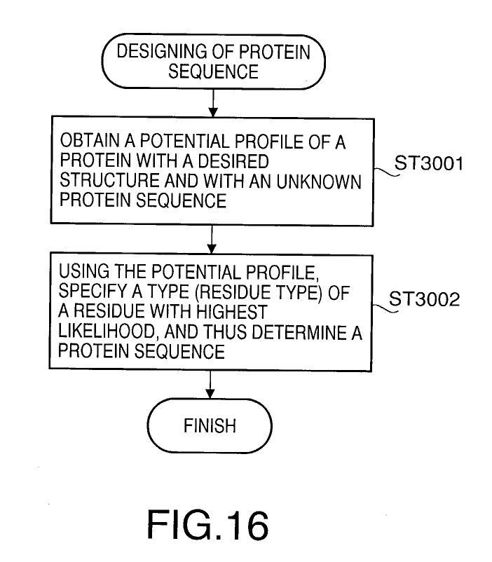

Procedures for designing a protein sequence as described above are as illustrated in FIG. 16.

A potential profile of a protein with a desired structure and with an unknown protein sequence is obtained (ST3001).

Using the potential profile, a type (residue type) of a residue with the highest likelihood is specified at each residue position, and thus a protein sequence is determined (ST3002).

Embodiments of the present invention will be described specifically below with reference to FIGS. 1 to 9.

(First embodiment)

FIG. 1 is a block diagram illustrating a protein tertiary structure predicting apparatus of the first embodiment of the present invention.

Protein tertiary structure predicting apparatus 1 is primarily separated into data preparing section 2 that generates potential profiles from a learning data set and structure predicting section 3 that evaluates the compatibility of each of the potential profiles generated in the data preparing section and a prediction target protein to predict its tertiary structure.

In data preparing section 2, learning data storing section 4 stores residue types at each of positions in a sequence of each of proteins with known tertiary structures composing the learning data set, and protein tertiary structure data composed of three-dimensional coordinates of atoms in the residue.

Statistical processing section 5 reads the tertiary structure data from learning data set storing section 4, and using the data, calculates expansion coefficients aI. Expansion coefficient storing section 6 stores expansion coefficients aI that statistical processing section 5 calculates on the entire protein.

Potential profile generating section 7 generates a potential profile of a protein to be identified as each template based on expansion coefficients aI stored in expansion coefficient storing section 6 and protein tertiary structures stored in learning data set storing section 4, and stores the generated profile in potential profile storing section 8.

In structure predicting section 3, compatibility evaluating section 10 performs alignment of protein sequence data of a prediction target protein input from data input section 9, with a potential profile read from potential profile storing section 8 in data preparing section 2.

Then, with respect to all potential profiles, the optimal alignment is determined, and evaluation result output section 11 outputs evaluation results including protein tertiary structures, alignment results, and alignment scores of proteins providing higher alignment scores among the optimal alignment results.

Procedures will be described specifically below for predicting a protein tertiary structure using protein tertiary structure predicting apparatus 1 with the above configuration.

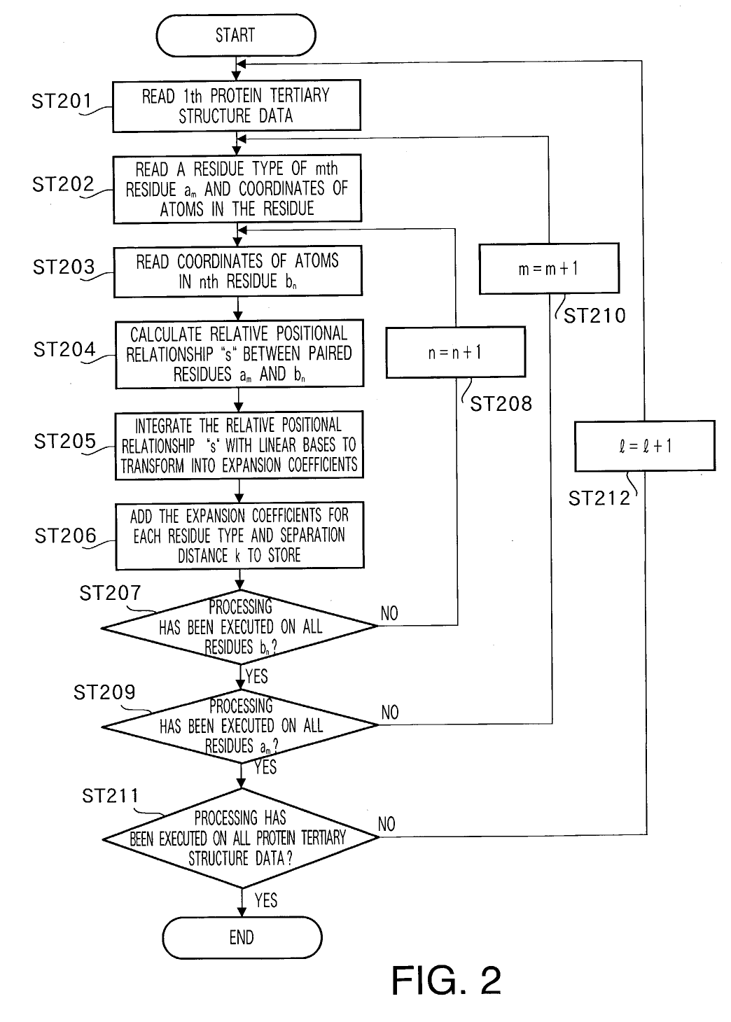

FIG. 2 is a flow diagram of statistical processing procedures in the statistical processing section in the protein tertiary structure predicting apparatus in the above embodiment.

Statistical processing section 5 reads first (default 1=1) protein tertiary structure data from learning data set storing section 4 (step (hereinafter referred to as ST) 201).

From the first protein tertiary structure data, the section 5 reads a residue type of an amino residue (hereinafter referred to as residue am) present at an mth (default m=1) position from one end and coordinates of atoms in the residue (ST202).

As an amino residue paring with the residue am, the section 5 reads an amino residue (hereinafter referred to residue bn) present at an nth (default n=1 and n≠m) position from one end and coordinates of atoms in the residue (ST203).

The section 5 calculates relative structural relationship “s” between paired residues am and bn (ST204). The relative structural relationship “s” is represented by six parameters consisting of residue-distance r, relative directions θ and φ, and relative orientations θe, φe, and φe.

The section 5 integrates the relative structural relationship “s” (represented by δ function) with linear bases to transform into expansion coefficients (ST205), and adds obtained expansion coefficients for each residue type (residue type of reside am) and separation distance k in a sequence (ST206).

Then, the section 5 determines whether or not the processing of ST203 to ST206 has been finished for all residues bn (ST207), and when the processing has not been finished, increments “n” by 1 to return to the processing of ST203.

In this way, the processing of ST203 to ST206 is repeated for next residue bn until “yes” is obtained in ST207. Thus, residue pairs are prepared of residue am and all residues bn available as pairs, their expansion coefficients are calculated, and expansion coefficients aI integrated for each residue type and separation distance k are obtained.

The section 5 determines whether or not expansion coefficients al have been obtained for all residues am (ST209), and when all the coefficients have not been obtained, increments “m” by 1 in ST210 to return to processing of ST202. In this way, the processing of ST202 to ST207 is repeated for next residue am until “yes” is obtained in ST209, and with respect to all residues am of the first protein tertiary structure, expansion coefficients integrated with expansion bases based on the relative structural relationship “s” are obtained.

When “yes” in ST209, the section 5 determines whether or not expansion coefficients have been obtained for all the protein tertiary structure data in the learning data set (ST210). When “no” in ST209, the section 5 increments “1” by 1 and returns to the processing of ST201. In this way, the processing of ST201 to ST209 is repeated for next protein tertiary structure data until “yes” is obtained in ST211. Thus, expansion coefficients aI integrated for each residue type “a” and separation distance “k” in a sequence are obtained and stored as one data group.

The processing in ST205 and ST206 will be described more specifically. In ST205, using the relative structural relationship “s” between paired residues am and bn as an observation sample, values of expansion bases at this point are obtained.

In this case, the statistics is calculated on samples with a residue-distance between paired residues am and bn less than or equal to predetermined value rmax. Thus extracting samples with residue-distances r in a predetermined range permits expansion using Legendre polynomial. Orthnormal gI (=gijklmn; γ,θ,φ,θe,φe, φe) associated with the relative structural relationship “s” with six parameters, i.e., residue-distance γ, directions θ and φ, and relative orientations θe,φe and φe used in the expansion is a product of orthogonal bases normalized by respective parameters, and is expressed as following equations (7-a) and (7-b).

gᵢⱼₖₗₘₙ(γ,θ,φ,θe,φe,φe)=Pi(z)Yⱼₖ(θ,φ)sin nθeYₗₘ(φ,φ) (7-a)

gᵢⱼₖₗₘₙ(γ,θ,φ,θe,φe,φe)=Pi(z)Yⱼₖ(θ,φ)cos nθeYₗₘ(φ,φ) (7-b)

That is, the transformed quotient the Legendre Polynomial divided by radius component r is used for residue-distance r when the distance is within the predetermined range, spherical harmonics are used for direction components θ and φ and orientation components θe and φe, and trigonometric functions are used for orientation component φe.

Thus, expansion coefficients aI are obtained by integrating relative structural relationship “s” as δ function in ST205, and further integrating the resultant for each residue “a” and separation distance k in ST206.

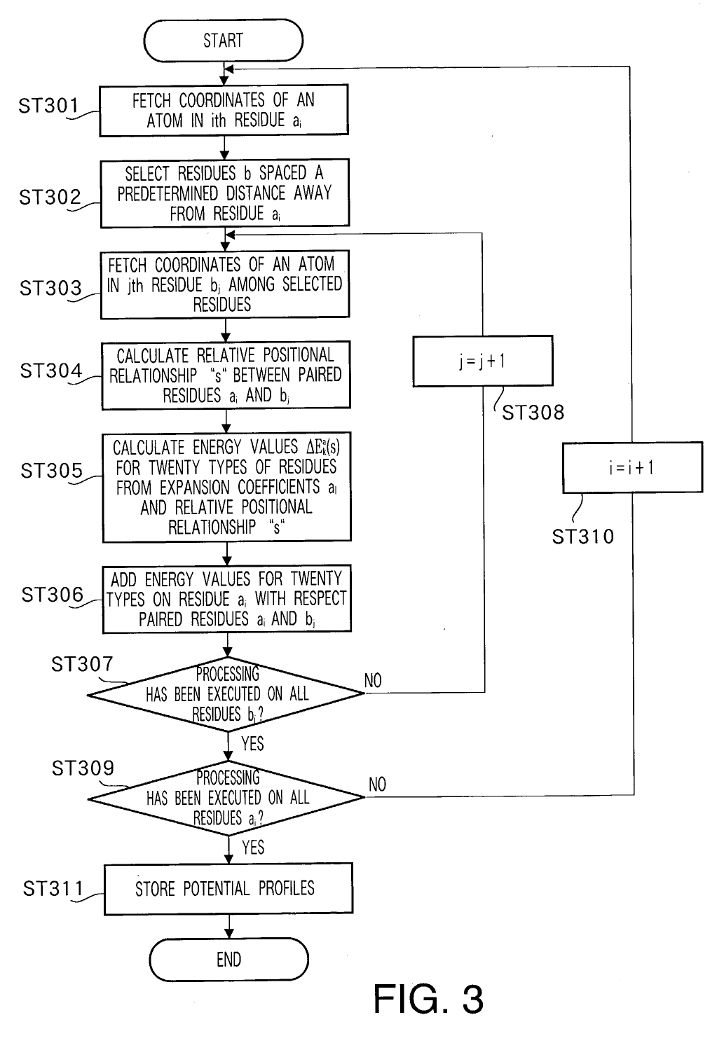

FIG. 3 is a flow diagram illustrating procedures of generating a potential profile in the potential profile generating section in the protein tertiary structure predicting apparatus in the above embodiment. The flow diagram illustrates procedures for generating a protein potential profile from a protein with a known tertiary structure.

Potential profile generating section 7 reads each item of tertiary structure data from learning data set storing section 4, and generates a protein potential profile as described below to store in potential profile storing section 8.

In addition, in this example, a potential profile is generated from protein tertiary structure data composing the learning data set. However, it may be possible to generate a potential profile based on tertiary structure data of a protein with a known tertiary structure different from those in the learning data set.

It is not required to generate potential profiles of all the proteins included in the learning data set.

Potential profile generating section 7 fetches coordinates of an atom in residue ai present at an ith (default i=1) from one end of protein tertiary structure data (ST301).

Next, potential profile generating section 7 selects residues spaced less than or equal to predetermined residue-distance rmax away from residue ai from the protein tertiary structure data (ST302), fetches coordinates of an atom in residue bj to be paired present at an jth (default n=1) position starting from one closer to one end among selected residues (ST303), and calculates the relative structural relationship “s” between paired residues ai and bj (ST304). The relative structural relationship “s” is obtained in the same way as in ST204 illustrated in FIG. 2.

Potential profile generating section 7 calculates the energy value ΔEak(s) for each of twenty types of residues ai with respect to residue pair ai and bi from the relative structural relationship “s” calculated in ST304 and frequency distribution fak(s) restored from expansion coefficients aI read from expansion coefficient storing section 6 (ST305). Then, potential profile generating section 7 adds the energy value ΔEak(s) calculated in ST305 for each residue type (ST306).

The section 7 determines whether or not energy values ΔEak(s) have been calculated for all residues bj selected in ST302 (ST307). When “no”, the section 7 increments “j” by 1 (ST303) and returns to the processing of ST303. Thus, until “yes” is obtained in ST307, in other words, until energy values ΔEak(s) are calculated for all residue pairs ai and bi and are added for each residue type, the processing of ST303 to ST306 is repeated.

The sum of energy values for each residue type in ST306 is an energy value (mean-force potential) Pia of residue ai at the ith residue position in tertiary structure C, and is expressed by following equation (8).

Pᵢₐ=ΣⱼΔE^{a}ᵢ₋ⱼ(Sᵢⱼ) (8)

Further, the section 7 determines whether or not the mean-force potentials Pia have been obtained for all residues ai of the protein tertiary structure data (ST309), and when “no”, increments “i” by 1 and returns to processing of ST310. Then, until “yes” is obtained in ST309, the section 7 repeats the processing of ST301 to ST307, and obtains mean-force potentials Pia for all the residues ai in the protein tertiary structure data.

Then, the section 7 stores the mean-force potentials Pia as a potential profile of a protein with the known tertiary structure in the potential profile storing section 8 (ST311), and finishes the processing.

As described above, using potential profiles generated in data preparing section 2 and stored in potential profile storing section 8, prediction of protein tertiary structure is executed as described below in structure predicting section 3.

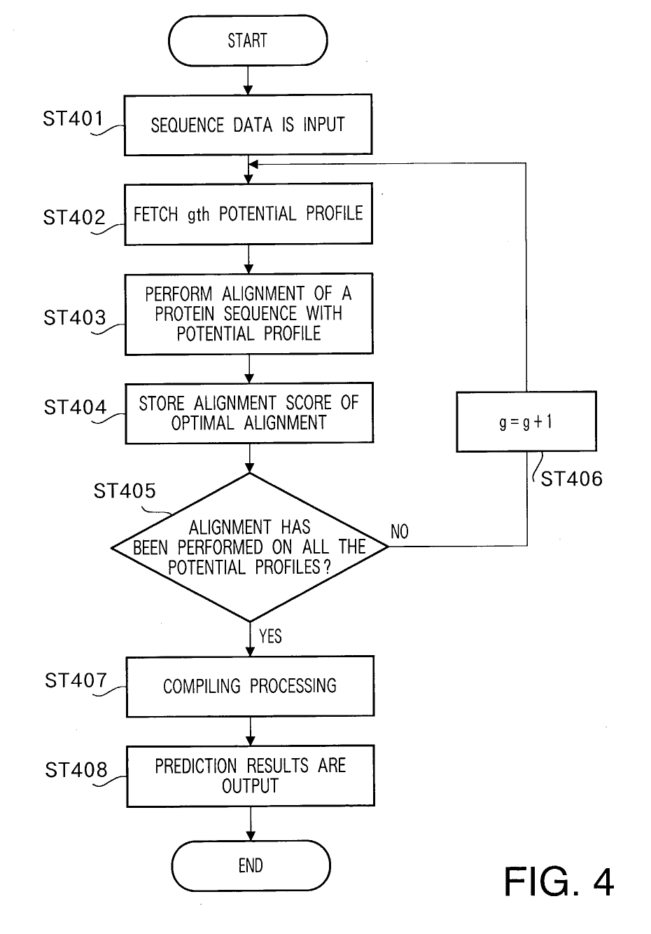

FIG. 4 is a flow diagram illustrating procedures of evaluating the compatibility in the compatibility evaluating section in the protein tertiary structure predicting apparatus in the above embodiment.

In structure predicting section 3, when protein sequence data of a prediction target protein is input from data input section 9 (ST401), compatibility evaluating section 10 fetches a gth (default g=1) potential profile (ST402).

Compatibility evaluating section 10 performs alignment of a protein sequence with the potential profile (ST403). The alignment is performed according to the dynamic programming. When the alignment is finished, an alignment score is stored of the optimal alignment of the gth potential profile (ST404).

The section 10 determines whether or not the alignment has been performed on all the potential profiles (ST405), and when “no”, increments “g” by 1 (ST406) and returns to ST402.

Thus, until “yes” is obtained in ST405, the section 10 repeats the processing of ST402 to ST404 for next potential profile, performs the alignment of the protein sequence with each of all the potential profiles, and stores the results.

When “yes” in ST405, compatibility evaluating section 10 compiles stored alignment results of all the potential profiles (ST407), and outputs potential profiles with higher alignment scores (for example, ten profiles in descending order of score) and alignment results with the protein sequence through evaluation result output section 11 (ST408). Users using the protein tertiary structure prediction method select tertiary structures suitable in biology and chemistry from among a plurality of alignment results, and based on them, are of modeling a protein tertiary structure to be predicted.

The alignment of a protein sequence with potential profile performed in ST403 will be described. Among dynamic programming algorithms used in the alignment, although a general dynamic programming algorithm using a subtraction method is also available, a dynamic programming algorithm using a continuation bonus method capable of obtaining optimal alignment with high accuracy will be described herein.

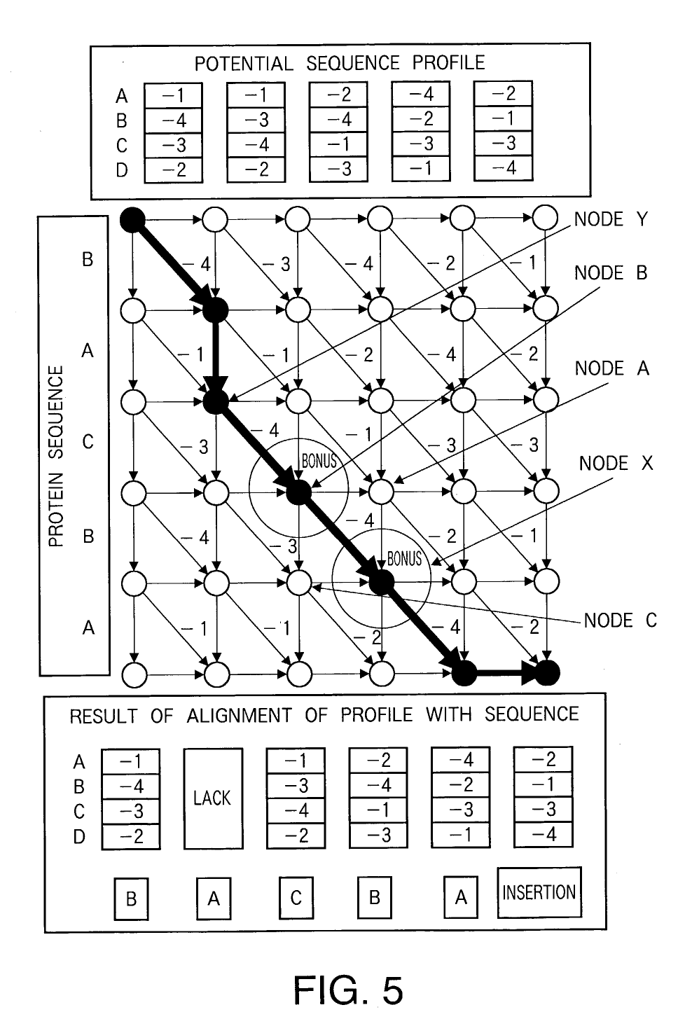

The details of the dynamic programming algorithm in calculating optimal alignment will be described. FIG. 5 is a diagram to explain a method of adding a bonus in alignment in the protein tertiary structure predicting apparatus in the above embodiment.

In FIG. 5, the horizontal direction indicates a direction of potential profile, while the vertical direction indicates a direction of protein sequence for alignment.

Accordingly, each arrow in a downward slanting direction indicates that an energy value of a position of the potential profile directly above the arrow matches a residue of a sequence to the left of the arrow. In this case, a value of the arrow indicates an energy value of a profile corresponding to the residue in the sequence to the left of the arrow. Arrows in a downward direction and in a rightward direction respectively indicate lack and insertion in the sequence.

An energy value of a profile directly above the arrow corresponding to a residue to the left of the arrow is substituted into each of all the arrows in a right-downward direction as a local value, and subtraction values corresponding to gap are substituted into arrows in the downward and rightward arrows.

A numeral value of each of the arrows in the right-downward direction in FIG. 5 indicates an energy value substituted into the arrow. Thus, a path with the most excellent value (path on which the sum of arrow values is the most excellent) is obtained from an upper left node to a lower right node, whereby residues in the sequence corresponding to the path are aligned with positions of the potential profile.

The algorithm selects, in each node to which arrows gather, the most excellent value (total sum of local values of all the arrows on the path) of a path led to the node among paths merging with the node, and thereby obtains a path finally selected in the lower right node as an optimal path.

The dynamic programming algorithm is not established unless there is a condition that a value of a path from the left upper node to a node is not dependent on another path beyond the node. It is because a path is selected in each node according to value of the path led to the node. Accordingly, in a method of using pairwise potentials, since an evaluation value on each path is determined as long as the entire alignment is determined, the dynamic programming is not applied.

In the present invention, in addition to the gap subtraction method where points are subtracted for insertion and lack, the continuation bonus method is combined which is a new method where points are added for portions with no gap, i.e., a path on which slant arrows are present continuously.

When a continuation bonus value is increased and a value to be subtracted for a gap is increased according to this method, the alignment is performed so as not to add a gap as possible and to shorten a length of a gap.

In other words, on the path according to the dynamic programming, the continuation bonus is added when paths are continuous of arrows in the right downward direction, which indicates residues match with portions of the structure.

More specifically, in FIG. 5, among paths merging with node X (paths from node A, B and C), when a path of an arrow in a right downward direction from node Y is selected in node B, since on a path from node B to node X the matching is continuous, the bonus is added in node B, and a path is selected in node X.

As described above, using both the continuation bonus and gap penalty where a point is subtracted for a path of a vertical or horizontal arrow results in an advantage that a length of a gap is controlled as compared with the case of using only the continuation bonus method.

In addition, a penalty score or bonus score may be one-tenth to ten times the average energy value of a residue in the potential profile.

Compatibility evaluating section 10 aligns a protein sequence of a prediction target protein with the potential profile according to the dynamic programming, and thereby obtains the optimal alignment. In other words, the mean-force potential is Pi(aj) when jth residue aj on the sequence S is correspondent with an ith residue position in the tertiary structure C.

At this point, since the alignment is not stabilized only by Pi(aj), P′ia is calculated according to following equation (9), and is used instead of mean-force potential Pi(aj).

P′i(aj)=Σ_{k=-N}^{k=N}Pi+k(aj+k) (9)

In other words, instead of using a local compatibility evaluation value at the jth residue position, a vicinity compatibility evaluation value is used in alignment that is an average value of local compatibility evaluation values of the residue at the jth residue position and N residues consecutive from the position.

In this way, in alignment, when a residue is correspondent with a place (residue position), evaluation values are given of when residues in the vicinity of the residue position are matched to the vicinity of the place (residue position). Therefore, even when a residue that is not suitable for the structure is locally inserted, the insertion of the residue that is not suitable for the structure is permitted as long as the vicinity evaluation value of the residue is high.

As a result, it is possible to prevent the local compatibility evaluation value from deteriorating and to stabilize the alignment.

Herein, a value of N ranges, for example, from 2 to 10. Further, the vicinity compatibility evaluation takes account of the correspondence between segments in the structure (or sequence), and copes with lengths required for secondary structure formation and domain formation of a protein. Therefore, it is possible to cope with the strict structure of a pertinent portion, and to evaluate the local compatibility with accuracy.

In the tertiary structure prediction by the protein tertiary structure predicting apparatus according to this embodiment as described above, the relative structural relationship “s” between paired residues is represented by multidimensional parameters in ST204 illustrated in ST204 in statistical processing in statistical processing section 5 in data preparing section 2.

Further, in potential profile generating section 7, the relative structural relationship between paired residues ai and bj in ST304 illustrated in ST304 is represented by multidimensional parameters.

Furthermore, in ST305, an energy value ΔEak(s) is calculated which is dependent only on the potential of residue ai and which has an average of potentials of residues bj available as a pair, for each of twenty residues, from expansion coefficients aI and relative structural relationship S.

Thus, in this embodiment, in the tertiary structure prediction using mean-force potentials, multidimensional singleton potentials are used. It is thereby possible to use fast dynamic programming to obtain the optimal alignment of a protein sequence with a tertiary structure in compatibility evaluating section 10 in tertiary structure predicting section 3, and to remarkably improve accuracy in identification in applying to structure identification.

Further, to evaluate performance of mean-force potential, identification experiments were performed of whether a protein sequence identifies its tertiary structure as a structure with the highest compatibility (on the premise that no gap is present when the sequence is aligned with the tertiary structure).

The result of the experiments showed when multidimensional mean-force potentials are used, there is no difference in performance in identification ratio between the case of using pairwise potentials based on both residue types of two residues contributing to the potentials and the case of using singleton potentials based on only a residue type of one residue.

In the case of one-dimensional potential based on only a distance, it was proved that using pairwise potentials has a higher identification ratio than using singleton potentials.

Further, is was confirmed that when gaps are permitted in alignment of a tertiary structure with a protein sequence in compatibility evaluating section 10, using singleton potentials is capable of obtaining the optimal alignment according to generally known dynamic programming, and the alignment score in such alignment can be used as an evaluation scale in compatibility.

In other words, the protein tertiary structure prediction method with adequate performance has been implemented for the first time by using multidimensional singleton mean-force potentials.

Further, in this embodiment, by using singleton potentials, the local compatibility evaluation value (Pia), which is for each residue in each residue position in a protein tertiary structure, can be determined independently of a sequence whose tertiary structure is to be predicted or alignment with the sequence.

Accordingly, potential profiles are determined in advance for protein tertiary structures when data preparing section 2 selects such a plurality of protein tertiary structures as a data set.

In structure predicting section 3, when data input section 9 provides a protein sequence of a protein whose tertiary structure is to be predicted to compatibility evaluating section 10, it is not required to regenerate potential profiles, and it is made possible to predict the tertiary structure only by performing alignment of a given sequence with stored profiles.

Accordingly, it is not necessary for all of protein tertiary structure predicting apparatuses 1 to comprise learning data set storing section 4, statistical processing section 5, expansion coefficient storing section 6 and potential profile generating section 7 in data preparing section 2.

In other words, using only structure predicting section 3 (or potential profile storing section 8), it is possible to perform alignment using potential profiles generated in another apparatus, and to output evaluation results.

This embodiment adopts so-called continuation bonus in which good evaluation values are added corresponding to a length of a portion without a gap, as illustrated in FIG. 5, in compatibility evaluation by the dynamic programming.

It is thereby possible to obtain stable compatibility evaluation scales even when lengths are extremely different between a protein sequence and that of a tertiary structure.

Further, the local compatibility evaluation value in the correspondence relationship between an ith residue in a protein sequence and a jth residue in a tertiary structure of a template protein is replaced with an average value of local compatibility evaluation values of a plurality of adjacent residues.

Such averaging processing introduces compatibility of adjacent residues in the vicinity, and thereby provides stable a local compatibility evaluation value even when the local compatibility evaluation value is low (which often occurs in protein), and as a result, the alignment is stabilized.

The experimental results showed the enormous effect, and it is possible to obtain similar alignment always independently of how to give a penalty to a gap. This evaluation method enables the optimal alignment to be obtained using the dynamic programming while permitting gaps in a sequence.

The present invention is not limited to the above embodiment. As is apparent to those skilled in the art, the present invention is capable of being carried into practice using a program according to techniques as described in the above embodiment, commercially available digital computer with the program and microprocessor. Further, as is apparent to those skilled in the art, the present invention includes computer programs generated by those skilled in the art based on techniques as described in the above embodiment.

Further, the scope of present invention includes computer program products that are media including orders for use in generating a program for a computer to implement the present invention. These media include, but not limited to, disks such as a flexible disk, optical disk, CD-ROM and magnetic disk, ROM, RAM, EPROM, EEPROM, magnetic-optical card, memory card and DVD.

(Second Embodiment)

In order to design artificial proteins with specific functions, it is required to design a tertiary structure appropriate for a target protein function, and to determine a protein sequence likely having the tertiary structure. This embodiment provides a method of automatically designing a protein sequence of a protein with the highest probability of having a tertiary structure that is designed as a target protein.

This embodiment of the present invention will be described below with reference to FIGS. 7 to 9.

In designing an artificial protein, in order to design a protein sequence of a protein having a target tertiary structure, a potential profile of the target tertiary structure is generated using the singleton potentials previously described, and with respect to each residue position, a residue type having the highest mean-force potential is determined as a residue type in the position.

The protein having a thus designed protein sequence has the high possibility of having a tertiary structure extremely similar to the designed target tertiary structure.

FIG. 7 is a block diagram illustrating a protein sequence designing apparatus according to the second embodiment of the present invention.

Protein sequence designing apparatus 22 has a configuration almost the same as that of the apparatus illustrated in FIG. 1. In the apparatus in FIG. 7, sections common to the apparatus in FIG. 1 are assigned the same reference numerals.

Protein sequence designing apparatus 22 in FIG. 7 is comprised of data preparing section 2 that generates potential profiles from a learning data set, and protein sequence generating section 32 that generates a protein sequence based on the potential profiles generated in data preparing section 2.

In data preparing section 2, potential profile generating section 7 generates potential profiles using multidimensional singleton potentials according to procedures as described in the first embodiment, and potential profile storing section 8 stores the generated potential profiles.

Protein sequence generating section 32 has protein sequence determining section 33 and protein sequence output section 34.

Protein sequence determining section 33 has selecting section 601. Selecting section 601 selects a residue type having the highest mean-force potential in each residue position.

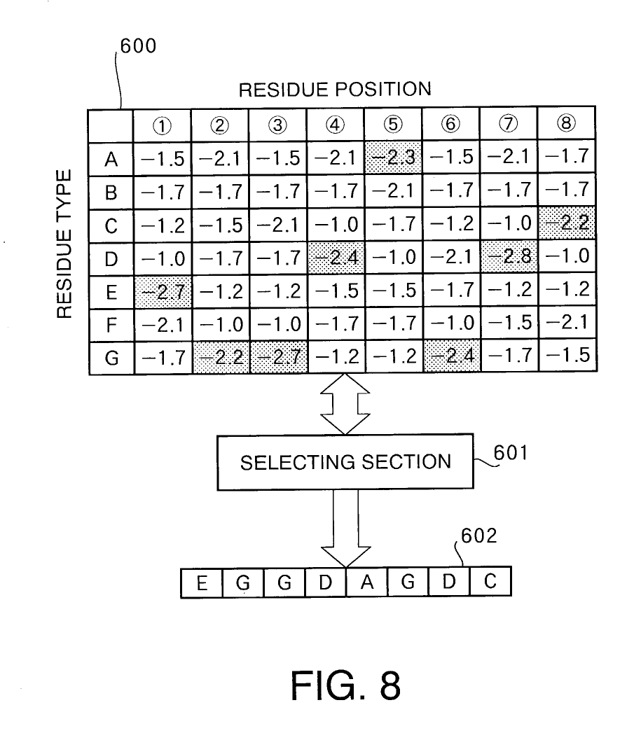

FIG. 8 illustrates an example of selection in selecting section 601.

Potential profile 600 illustrated at an upper side in FIG. 8 shows mean-force potentials of residue types A to G in residue positions \mathring{1} to \mathring{8}.

Selecting section 601 selects a residue type with the lowest energy value (residue type with the highest likelihood) in each residue position. Designed protein sequence 602 is thus obtained. The designed protein sequence is output from protein sequence output section 34 in FIG. 7.

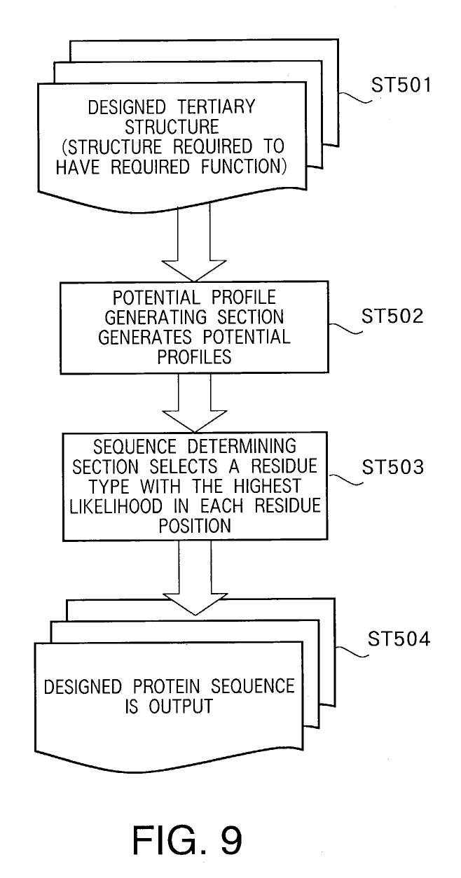

Procedures as described above are as illustrated in FIG. 9.

In other words, data is prepared (ST501) which is associated with a designed protein tertiary structure (structure required for having a required function), and potential profile generating section 7 generates potential profiles (ST502).

Next, protein sequence determining section 33 selects a residue type with the highest likelihood in each residue position (ST503), and outputs the designed protein sequence (ST504).

As described above, according to the present invention, in the protein tertiary structure prediction using mean-force potentials, multidimensional singleton potentials are used. It is thereby possible to use dynamic programming that is generally used and works fast to obtain the optimal alignment of a protein sequence of a protein with an unknown structure with a tertiary structure of a protein to be identified, and to remarkably improve accuracy in structure identification.

Further, similarly, it is possible to obtain a protein sequence of a protein with a designed structure rapidly and accurately by the simplified method.

This application is based on the Japanese Patent Applications No. 2000-360684 filed on Nov. 28, 2000, and No. 2001-355309 filed on Nov. 20, 2001, entire contents of which are expressly incorporated by reference herein.

### INDUSTRIAL APPLICABILITY

The present invention is capable of being used in protein tertiary structure prediction and protein sequence designing.

1. A potential profile generating method for obtaining energy values for each residue type in each of residue positions in a tertiary structure that is already known of a protein and generating a potential profile composed of information of the energy values for each residue type in each of residue positions, said method using multidimensional singleton potentials as potentials used in obtaining the energy values, wherein each of the singleton potentials is dependent on a multidimensional relative structural relationship and on a residue type of one of residues of a residue pair.

2. A potential profile generating method comprising:
calculating an energy value of a residue at a residue position in a protein with a known tertiary structure using multidimensional singleton potentials dependent on a relative structural relationship of residues of a residue pair and on only a type of one of residues of the residue pair, while assuming each of the plurality of residues forms a residue pair with each of the other residues and further assuming a type of the residue as one of the residues;
obtaining a mean-force potential on the residue by adding energy values for all pairs calculated using the multidimensional singleton potentials, changing a type of the residue at the residue position to another type, and obtaining mean-force potentials for each of types of changed residues; and
executing processing for obtaining the mean-force potentials using singleton potentials for residues at the other positions in the protein with the known tertiary structure, and generating a potential profile of the protein with the known tertiary structure.

3. A protein tertiary structure prediction method comprising:
preparing a plurality of template proteins with known tertiary structures, and obtaining potential profiles in advance for the template proteins using the method according to claim 1 or 2;
applying a protein sequence of a protein whose tertiary structure is to be predicted to each of the plurality of template proteins with known tertiary structures to obtain optimal alignment, and obtaining an evaluation value that is a criterion to evaluate a degree of the optimal alignment for each of the template proteins; and
comparing the valuation value to each other with respect to the template proteins, estimating that a tertiary structure of a template protein with the highest evaluation value is similar to a tertiary structure of the protein sequence of the protein, and thereby predicting a protein tertiary structure.

4. A protein sequence designing method comprising:
generating a potential profile of a protein having a desired structure and an unknown protein sequence using the method according to claim 1 or 2; and
specifying a type (residue type) of a residue with the highest likelihood in each residue position using the potential profile, and thus determining a protein sequence.

5. A protein tertiary structure prediction method comprising:
obtaining a frequency distribution of multidimensional relative structural relationship of each of all residue pairs of each protein from tertiary structure data of a plurality of proteins with known tertiary structures;
calculating, by using the frequency distribution, an energy value based on multidimensional singleton potentials dependent on the multidimensional relative structural relationship and only on a residue type of one of residues of a residue pair from tertiary structure data of a plurality of template proteins to be identified with known tertiary structures, and adding energy values for each residue type to obtain mean-force potentials, with respect to each residue position of residue pairs of each template protein; and
evaluating a compatibility between a protein sequence of a prediction target protein with an unknown tertiary structure and a protein sequence of each template protein using the calculated mean-force potentials, and searching for a template protein having a tertiary structure similar to that of the prediction target protein.

6. The method according to claim 5, wherein the multidimensional relative structural relationship includes at least two selected from among a distance between residues of a residue pair, a relative direction and relative orientation.

7. The method according to claim 6, wherein the multidimensional relative structural relationship is a three-dimensional relationship comprising a distance r between residues of a residue pair, and directions θ and φ.

8. The method according to claim 5, wherein when the frequency distribution of relative structural relationship is obtained, multidimensional frequency statistical processing is executed using an information compressing operation using Fourier expansion.

9. The method according to claim 8, wherein in the information compressing operation using Fourier expansion, as linear bases of a distance direction component, Legendre Polynomial that is orthonormal in a designated area is used.

10. The method according to claim 5, wherein in obtaining the mean-force potentials for each residue type, the mean-force potentials are obtained for each of the template proteins to generate potential profiles, and in evaluating a compatibility, compatibility between a protein sequence of the prediction target protein and each of the potential profiles is evaluated.

11. The method according to claim 10, wherein in evaluating a compatibility between a protein sequence of the prediction target protein and each of the potential profiles, an average value is used as a compatibility evaluation value, the average value of mean-force potentials of residues at a residue position of a template protein corresponding to a residue type in the protein sequence and at a plurality of residue positions in the vicinity of the residue position.

12. The method according to claim 10, wherein in evaluating a compatibility between a protein sequence of the prediction target protein and each of the potential profiles, optimal alignment of the potential profile with the residue is obtained using dynamic programming, and compatibility is evaluated based on an alignment score of the optimal alignment.

13. The method according to claim 12, wherein in the dynamic programming, a bonus is added corresponding to a length of a consecutive matching region where insertion or lack is not present.

14. A potential profile generating method for evaluating a compatibility between a protein sequence of a prediction target protein with an unknown structure and each of template proteins to search for a template protein with a tertiary structure similar to that of the prediction target protein, comprising:
obtaining a frequency distribution of multidimensional relative structural relationship of each of all residue pairs of each protein from tertiary structure data of a plurality of proteins with known tertiary structures; and
calculating, by using the frequency distribution, an energy value based on multidimensional singleton potentials dependent on the multidimensional relative structural relationship and only on a residue type of one of residues of a residue pair from tertiary structure data of a plurality of template proteins to be identified with known tertiary structures, adding energy values for each residue type to obtain mean-force potentials, with respect to each residue position of residue pairs of each template protein, and obtaining mean-force potentials for each template protein to generate potential profiles.

15. A protein tertiary structure prediction method comprising:
evaluating a compatibility between a potential profile using multidimensional singleton potentials dependent on a multidimensional relative structural relationship between residues obtained from a frequency distribution of known protein tertiary structures and only on a residue type of one of residues of a residue pair, and a protein sequence of a prediction target protein with an unknown tertiary structure; and
searching for a template protein having a tertiary structure similar to that of the prediction target protein based on the evaluation result.

16. A protein tertiary structure predicting apparatus comprising:
a frequency distribution calculating section that calculates a frequency distribution of multidimensional relative structural relationship of each of all residue pairs of each protein from tertiary structure data of a plurality of proteins with known tertiary structures;
a potential calculating section which with respect to each residue position of residue pairs of each template protein, calculates an energy value based on multidimensional singleton potentials dependent on the multidimensional relative structural relationship and only on a residue type of one of residues of a residue pair, from tertiary structure data of a plurality of template proteins to be identified with known tertiary structures, using the frequency distribution obtained in the frequency distribution calculating section, and adds energy values for each residue type to calculate mean-force potentials; and
a compatibility evaluating section that evaluates a compatibility between a reside sequence of a prediction target protein with an unknown structure and each of the template proteins using the mean-force potentials obtained in the potential calculating section.

17. A potential profile generating apparatus for evaluating a compatibility between a protein sequence of a prediction target protein with an unknown structure and each of template proteins to search for a template protein with a tertiary structure similar to that of the prediction target protein, said apparatus comprising:
a distribution calculating section that calculates a frequency distribution of multidimensional relative structural relationship of each of all residue pairs of each protein from tertiary structure data of a plurality of proteins with known tertiary structures; and
a potential profile generating section which with respect to each residue position of residue pairs of each template protein, calculates an energy value based on multidimensional singleton potentials dependent on the multidimensional relative structural relationship and only on a residue type of one of residues of a residue pair, from tertiary structure data of a plurality of template proteins to be identified with known tertiary structures, using the frequency distribution, adds energy values for each residue type to obtain mean-force potentials, and obtains mean-force potentials for each to generate a potential profile.

18. A protein tertiary structure predicting apparatus comprising:
a compatibility evaluating section that evaluates a compatibility between a potential profile using multidimensional singleton potentials dependent on a multidimensional relative structural relationship between residues obtained from a frequency distribution of known protein tertiary structures and only on a residue type of one of residues of a residue pair, and a protein sequence of a prediction target protein with an unknown structure; and
a similar tertiary structure searching section that searches for a template protein having a tertiary structure similar to that of the prediction target protein based on the evaluation result.

19. A program for making a computer execute the procedures of:
calculating a frequency distribution of multidimensional relative structural relationship of each of all residue pairs of each protein from tertiary structure data of a plurality of proteins with known tertiary structures;
calculating, with respect to each residue position of residue pairs of each protein, an energy value based on multidimensional singleton potentials dependent on the multidimensional relative structural relationship and only on a residue type of one of residues of a residue pair, from tertiary structure data of a plurality of template proteins to be identified with known tertiary structures using the frequency distribution, and adding energy values for each residue type to obtain mean-force potentials; and
evaluating a compatibility between a protein sequence of a prediction target protein with an unknown structure and each of the template proteins using stored mean-force potentials to search for a template protein likely having a tertiary structure similar to that of the template protein.

20. A program which evaluates a compatibility between a protein sequence of a prediction target protein with an unknown structure and each of template proteins to search for a template protein with a tertiary structure similar to that of the prediction target protein, and which makes a computer execute the procedures of:
calculating a frequency distribution of multidimensional relative structural relationship of each of all residue pairs of each protein from tertiary structure data of a plurality of proteins with known tertiary structures; and
calculating, with respect to each residue position of residue pairs of each protein, an energy value based on multidimensional singleton potentials dependent on the multidimensional relative structural relationship and only on a residue type of one of residues of a residue pair, from tertiary structure data of a plurality of template proteins to be identified with known tertiary structures using the frequency distribution, adding energy values for each residue type to obtain mean-force potentials, and obtaining mean-force potentials for each template protein to generate potential profiles.

21. A program for making a computer execute the procedures of:
evaluating a compatibility between a potential profile using multidimensional singleton potentials dependent on a multidimensional relative structural relationship between residues obtained from a frequency distribution of known protein tertiary structures and only on a residue type of one of residues of a residue pair, and a protein sequence of a prediction target protein with an unknown structure; and
searching for a template protein having a tertiary structure similar to that of the prediction target protein based on the evaluation result.

22. A computer readable storage medium storing a program for making a computer execute the procedures of:
calculating a frequency distribution of multidimensional relative structural relationship of each of all residue pairs of each protein from tertiary structure data of a plurality of proteins with known tertiary structures;
calculating, with respect to each residue position of residue pairs of each protein, an energy value based on multidimensional singleton potentials dependent on the multidimensional relative structural relationship and only on a residue type of one of residues of a residue pair, from tertiary structure data of a plurality of template proteins to be identified with known tertiary structures using the frequency distribution, and adding energy values for each residue type to obtain mean-force potentials; and
evaluating a compatibility between a protein sequence of a prediction target protein with an unknown structure and each of the template proteins using stored mean-force potentials to search for a template protein likely having a tertiary structure similar to that of the template protein.

23. A computer readable storage medium storing a program which evaluates a compatibility between a protein sequence of a prediction target protein with an unknown structure and each of template proteins to search for a template protein with a tertiary structure similar to that of the prediction target protein, and which makes a computer execute the procedures of:
calculating a frequency distribution of multidimensional relative structural relationship of each of all residue pairs of each protein from tertiary structure data of a plurality of proteins with known tertiary structures; and
calculating, with respect to each residue position of residue pairs of each protein, an energy value based on multidimensional singleton potentials dependent on the multidimensional relative structural relationship and only on a residue type of one of residues of a residue pair, from tertiary structure data of a plurality of template proteins to be identified with known tertiary structures using the frequency distribution and adding energy values for each residue type to obtain mean-force potentials, and obtaining mean-force potentials for each template protein to generate potential profiles.

24. A computer readable storage medium storing a program for making a computer execute the procedures of:
evaluating a compatibility between a potential profile using multidimensional singleton potentials dependent on a multidimensional relative structural relationship between residues obtained from a frequency distribution of known protein tertiary structures and only on a residue type of one of residues of a residue pair, and a protein sequence of a prediction target protein with an unknown structure; and
searching for a template protein having a tertiary structure similar to that of the prediction target protein based on the evaluation result.

25. A protein sequence designing method comprising:
obtaining a frequency distribution of multidimensional relative structural relationship of each of all residue pairs of each protein from tertiary structure data of a plurality of proteins with known tertiary structures;
calculating, by using the frequency distribution, an energy value based on multidimensional singleton potentials dependent on the multidimensional relative structural relationship and only on a residue type of one of residues of a residue from tertiary structure data of a plurality of template proteins to be identified with known tertiary structures, and adding energy values for each residue type to obtain mean-force potentials, with respect to each residue position of residue pairs of each template protein; and
specifying a type (reside type) of a residue with the highest likelihood in each residue position using obtained mean-force potentials, and thereby determining a protein sequence.

26. The method according to claim 25, wherein the multidimensional relative structural relationship includes at least two selected from among a distance between residues of a residue pair, a relative direction and relative orientation.

27. The method according to claim 25, wherein the multidimensional relative structural relationship is a three-dimensional relationship comprising a distance r between residues of a residue pair, and directions θ and φ.

28. The method according to claim 25, wherein when a frequency distribution of relative structural relationship is obtained, multidimensional frequency statistical processing is executed using an information compressing operation using Fourier expansion.

29. The method according to claim 28, wherein in the information compressing operation using Fourier expansion, as linear bases of a distance direction component, Legendre Polynomial that is orthonormal in a designated area is used.

30. A protein sequence designing apparatus comprising:
a frequency distribution calculating section that calculates a frequency distribution of multidimensional relative structural relationship of each of all residue pairs of each protein from tertiary structure data of a plurality of proteins with known tertiary structures;
a potential calculating section which with respect to each residue position of residue pairs of each template protein, calculates an energy value based on multidimensional singleton potentials dependent on the multidimensional relative structural relationship and only on a residue type of one of residues of a residue pair, from tertiary structure data of a plurality of template proteins to be identified with known tertiary structures, using the frequency distribution obtained in the frequency distribution calculating section, and adds energy values for each residue type to obtain mean-force potentials; and
a protein sequence determining section that specifies a type of a residue (residue type) with the highest likelihood in each residue position using the mean-force potentials calculated in the potential calculating section, and thereby determines a protein sequence.

31. A computer readable storage medium storing a program for making a computer execute a procedure of specifying a type of a residue (residue type) with the highest likelihood in each residue position to determine a protein sequence, using mean-force potentials obtained by using multidimensional singleton potentials dependent on a multidimensional relative structural relationship between residues obtained from a frequency distribution of known protein tertiary structures and only on a residue type of one of residues of a residue pair.
